SEQUENCE LISTING (29 sequences)

SEQ ID NO: 1 - protein, 10 aa, synthetic construct
  SITE 3: note = ornithine
  VARIANT 4: note = X can be any amino acid
  SITE 5: note = D-alanine
  VARIANT 6: note = X can be any amino acid
  SITE 8: note = D-serine
  VARIANT 9: note = glutamic acid, 3-methyl-glutamic acid or gamma-hydroxyglutamic acid
  SITE 10: note = kynurenine
  SITE 1..10: note = kynurenine is linked to the side chain of threonine
TGXXAXGSEX

SEQ ID NO: 2 - protein, 10 aa, synthetic construct
  SITE 3: note = ornithine
  VARIANT 4: note = X can be any amino acid
  SITE 5: note = D-alanine
  VARIANT 6: note = X can be any amino acid
  SITE 8: note = D-alanine
  VARIANT 9: note = gutamic acid, 3-methyl-glutamic acid, or gamma-hydroxyglutamic acid
  VARIANT 10: note = kynurenine or 4-chloro-kynurenine
  SITE 1..10: note = kynurenine is linked to the side chain of threonine
TGXXAXGAEX

SEQ ID NO: 3 - protein, 10 aa, synthetic construct
  MOD_RES 2: note = sarcosine
  VARIANT 4: note = X can be any amino acid
  SITE 5: note = D-lysine
  VARIANT 6: note = X can be any amino acid
  SITE 8: note = D-asparagine
  VARIANT 9: note = glutamic acid, 3-methyl-glutamic acid, or gamma-hydroxy-glutamic acid
  VARIANT 10: note = Ile or Val
  SITE 1..10: note = isoleucine or valine is linked to the side chain of threonine
TGAXKXGNEI

SEQ ID NO: 4 - protein, 10 aa, synthetic construct
  SITE 2: note = D-tryptophan
  VARIANT 3: note = X can be any amino acid
  VARIANT 4: note = X can be any amino acid
  SITE 5: note = D-4-hydroxyphenylglycine
  VARIANT 6: note = X can be any amino acid
  VARIANT 8: note = D-beta-hydroxy-asparagine or a phosphorylated form thereof
  VARIANT 9: note = glutamic acid, 3-methyl-glutamic acid, or gamma-hydroxy-glutamic acid
  VARIANT 10: note = tryptophan or Z-2,3-dehydrotryptophan
  SITE 1..10: note = tryptophan is linked to the side chain of threonine
TWXXXXGNEW

SEQ ID NO: 5 - protein, 9 aa, synthetic construct
  SITE 2: note = D-isoleucine
  VARIANT 3: note = X can be any amino acid
  VARIANT 4: note = X can be any amino acid
  SITE 7: note = D-glutamic acid
  VARIANT 8: note = glutamic acid, 3-methyl-glutamic acid, or gamma-hydroxy-glutamic acid
  MOD_RES 9: note = sarcosine
  SITE 1..9: note = sarcosine is linked to the side chain of threonine
TIXXPGEEG

SEQ ID NO: 6 - protein, 9 aa, synthetic construct
  SITE 1: note = diaminopimelic acid
  SITE 2: note = D-valine
  VARIANT 4: note = X can be any amino acid
  VARIANT 5: note = X can be any amino acid
  VARIANT 7: note = X can be any amino acid
  SITE 9: note = 4-methyl-proline
  SITE 1..9: note = proline is linked to the side chain of diaminopimelic acid
XVKXXGXVP

SEQ ID NO: 7 - protein, 10 aa, synthetic construct
  SITE 1: note = diaminopimelic acid
  SITE 2: note = D-pipecolic acid
  VARIANT 4: note = X can be any amino acid
  VARIANT 6: note = X can be any amino acid
  SITE 8: note = D-threonine
  SITE 1..10: note = proline is linked to the side chain of diaminopimelic acid
XXGXGXGTIP

SEQ ID NO: 8 - protein, 10 aa, synthetic construct
  SITE 1: note = diaminopimelic acid
  SITE 2: note = D-pipecolic acid
  VARIANT 3: note = X can be any amino acid
  VARIANT 4: note = X can be any amino acid
  VARIANT 6: note = X can be any amino acid
  SITE 8: note = D-diaminobutyric acid
  SITE 1..10: note = proline is linked to the side chain of diaminopimelic acid
XXXXGXGXVP

SEQ ID NO: 9 - protein, 10 aa, synthetic construct
  SITE 3: note = ornithine
  SITE 5: note = D-alanine
  VARIANT 6: note = X can be any amino acid
  SITE 8: note = D-serine
  VARIANT 9: note = glutamic acid, 3-methyl-glutamic acid, or gamma-hydroxy-glutamic acid
  SITE 10: note = kynurenine
  SITE 1..10: note = kynurenine is linked to the side chain of threonine
TGXDAXGSEX

SEQ ID NO: 10 - protein, 10 aa, synthetic construct
  SITE 3: note = ornithine
  SITE 5: note = D-alanine
  VARIANT 6: note = X can be any amino acid
  SITE 8: note = D-alanine
  VARIANT 9: note = glutamic acid, 3-methyl-glutamic acid, or gamma-hydroxy-glutamic acid
  SITE 10: note = 4-chloro-kynurenine
  SITE 1..10: note = kynurenine is linked to the side chain of threonine
TGXDAXGAEX

SEQ ID NO: 11 - protein, 10 aa, synthetic construct
  MOD_RES 2: note = sarcosine
  SITE 5: note = D-lysine
  VARIANT 6: note = X can be any amino acid
  SITE 8: note = D-asparagine
  VARIANT 9: note = glutamic acid, 3-methyl-glutamic acid, or gamma-hydroxy-glutamic acid
  VARIANT 10: note = Ile or Val
  SITE 1..10: note = isoleucine or valine is linked to the side chain of threonine
TGADKXGNEI

SEQ ID NO: 12 - protein, 10 aa, synthetic construct
  SITE 2: note = D-tryptophan
  VARIANT 3: note = X can be any amino acid
  SITE 5: note = D-4-hydroxyphenylglycine
  VARIANT 6: note = X can be any amino acid
  VARIANT 8: note = D-beta-hydroxy-asparagine or a phosphorylated form thereof
  VARIANT 9: note = glutamic acid, 3-methyl-glutamic acid, or gamma-hydroxy-glutamic acid
  VARIANT 10: note = tryptophan or Z-2,3-dehydrotryptophan
  SITE 1..10: note = tryptophan is linked to the side chain of threonine
TWXDXXGNEW

SEQ ID NO: 13 - protein, 10 aa, synthetic construct
  SITE 1: note = diaminopimelic acid
  SITE 2: note = D-pipecolic acid
  VARIANT 6: note = X can be any amino acid
  SITE 8: note = D-threonine
  SITE 1..10: note = proline is linked to the side chain of diaminopimelic acid
XXGDGXGTIP

SEQ ID NO: 14 - protein, 10 aa, synthetic construct
  SITE 1: note = diaminopimelic acid
  SITE 2: note = D-pipecolic acid
  VARIANT 3: note = X can be any amino acid
  VARIANT 6: note = X can be any amino acid
  SITE 8: note = D-diaminobutyric acid
  SITE 1..10: note = proline is linked to the side chain of diaminopimelic acid
XXXDGXGXVP

SEQ ID NO: 15 - protein, 10 aa, synthetic construct
  SITE 1: note = diaminopimelic acid
  SITE 2: note = D-pipecolic acid
  SITE 8: note = D-threonine
  SITE 1..10: note = proline is linked to the side chain of diaminopimelic acid
XXGDGSGTIP

SEQ ID NO: 16 - protein, 10 aa, synthetic construct
  SITE 1: note = diaminopimelic acid
  SITE 2: note = D-pipecolic acid
  SITE 6: note = homoserine
  SITE 8: note = D-threonine
  SITE 1..10: note = proline is linked to the side chain of diaminopimelic acid
XXGDGXGTIP

SEQ ID NO: 17 - protein, 13 aa, synthetic construct
  SITE 2: note = D-asparagine
  VARIANT 3: note = X can be any amino acid
  SITE 6: note = ornithine
  VARIANT 7: note = X can be any amino acid
  SITE 8: note = D-alanine
  VARIANT 9: note = X can be any amino acid
  SITE 11: note = D-serine
  VARIANT 12: note = glutamic acid, 3-methyl-glutamic acid, or gamma-hydroxy-glutamic acid
  SITE 13: note = kynurenine
  SITE 4..13: note = kynurenine is linked to the side chain of threonine
WNXTGXXAXGSEX

SEQ ID NO: 18 - protein, 13 aa, synthetic construct
  VARIANT 1: note = tryptophan or Z-2,3-dehydrotryptophan
  SITE 2: note = D-asparagine
  VARIANT 3: note = X can be any amino acid
  SITE 6: note = ornithine
  VARIANT 7: note = X can be any amino acid
  SITE 8: note = D-alanine
  VARIANT 9: note = X can be any amino acid
  SITE 11: note = D-alanine
  VARIANT 12: note = glutamic acid, 3-methyl-glutamic acid, or gamma-hydroxy-glutamic acid
  SITE 13: note = 4-chloro-kynurenine
  SITE 4..13: note = kynurenine is linked to the side chain of threonine
WNXTGXXAXGAEK

SEQ ID NO: 19 - protein, 13 aa, synthetic construct
  SITE 2: note = D-glutamic acid
  VARIANT 3: note = beta-hydroxy-asparagine or a phosphorylated form thereof
  MOD_RES 5: note = sarcosine
  VARIANT 7: note = X can be any amino acid
  SITE 8: note = D-lysine
  VARIANT 9: note = X can be any amino acid
  SITE 11: note = D-asparagine
  VARIANT 12: note = glutamic acid, 3-methyl-glutamic acid, or gamma-hydroxy-glutamic acid
  VARIANT 13: note = Ile or Val
  SITE 4..13: note = isoleucine or valine is linked to the side chain of threonine
WENTGAXKXGNEI

SEQ ID NO: 20 - protein, 11 aa, synthetic construct
  SITE 3: note = D-tryptophan
  VARIANT 4: note = X can be any amino acid
  VARIANT 5: note = X can be any amino acid
  SITE 6: note = D-4-hydroxyphenylglycine
  VARIANT 7: note = X can be any amino acid
  VARIANT 9: note = D-beta-hydroxy-asparagine or a phosphorylated form thereof
  VARIANT 10: note = glutamic acid, 3-methyl-glutamic acid, or gamma-hydroxy-glutamic acid
  VARIANT 11: note = tryptophan or Z-2,3-dehydrotryptophan
  SITE 2..11: note = tryptophan is linked to the side chain of threonine
STWXXXXGNEW

SEQ ID NO: 21 - protein, 12 aa, synthetic construct
  SITE 2: note = D-glutamic acid
  SITE 5: note = D-isoleucine
  VARIANT 6: note = X can be any amino acid
  VARIANT 7: note = X can be any amino acid
  SITE 10: note = D-glutamic acid
  VARIANT 11: note = glutamic acid, 3-methyl-glutamic acid, or gamma-hydroxy-glutamic acid
  MOD_RES 12: note = sarcosine
  SITE 4..12: note = sarcosine is linked to the side chain of threonine
AEYTIXXPGEEG

SEQ ID NO: 22 - protein, 10 aa, synthetic construct
  VARIANT 1: note = X can be any amino acid
  SITE 2: note = diaminopimelic acid
  SITE 3: note = D-valine
  VARIANT 5: note = X can be any amino acid
  VARIANT 6: note = X can be any amino acid
  VARIANT 8: note = X can be any amino acid
  SITE 10: note = 4-methyl-proline
  SITE 2..10: note = proline is linked to the side chain of diaminopimelic acid
XXVKXXGXVP

SEQ ID NO: 23 - protein, 11 aa, synthetic construct
  VARIANT 1: note = X can be any amino acid
  SITE 2: note = diaminopimelic acid
  SITE 3: note = D-pipecolic acid
  VARIANT 5: note = X can be any amino acid
  VARIANT 7: note = X can be any amino acid
  SITE 9: note = D-threonine
  SITE 2..11: note = proline is linked to the side chain of diaminopimelic acid
XXXGXGXGTIP

SEQ ID NO: 24 - protein, 11 aa, synthetic construct
  VARIANT 1: note = X can be any amino acid
  SITE 2: note = diaminopimelic acid
  SITE 3: note = D-pipecolic acid
  VARIANT 4: note = X can be any amino acid
  VARIANT 5: note = X can be any amino acid
  VARIANT 7: note = X can be any amino acid
  SITE 9: note = D-diaminobutyric acid
  SITE 2..11: note = proline is linked to the side chain of diaminopimelic acid
XXXXXGXGXVP

SEQ ID NO: 25 - protein, 11 aa, synthetic construct
  SITE 2: note = diaminopimelic acid
  SITE 3: note = D-pipecolic acid
  VARIANT 4: note = X can be any amino acid
  VARIANT 5: note = X can be any amino acid
  VARIANT 7: note = X can be any amino acid
  SITE 9: note = D-diaminobutyric acid
  SITE 2..11: note = proline is linked to the side chain of diaminopimelic acid
NXXXXGXGXVP

SEQ ID NO: 26 - protein, 11 aa, synthetic construct
  SITE 2: note = diaminopimelic acid
  SITE 3: note = D-pipecolic acid
  SITE 9: note = D-threonine
  SITE 2..11: note = proline is linked to the side chain of diaminopimelic acid
SXXGDGSGTIP

SEQ ID NO: 27 - protein, 11 aa, synthetic construct
  SITE 1: note = homoserine
  SITE 2: note = diaminopimelic acid
  SITE 3: note = D-pipecolic acid
  SITE 7: note = homoserine
  SITE 9: note = D-threonine
  SITE 2..11: note = proline is linked to the side chain of diaminopimelic acid
XXXGDGXGTIP

SEQ ID NO: 28 - protein, 11 aa, synthetic construct
  SITE 2: note = diaminopimelic acid
  SITE 3: note = D-pipecolic acid
  SITE 7: note = homoserine
  SITE 9: note = D-threonine
  SITE 2..11: note = proline is linked to the side chain of diaminopimelic acid
SXXGDGXGTIP

SEQ ID NO: 29 - protein, 11 aa, synthetic construct
  SITE 1: note = homoserine
  SITE 2: note = diaminopimelic acid
  SITE 3: note = D-pipecolic acid
  SITE 9: note = D-threonine
  SITE 2..11: note = proline is linked to the side chain of diaminopimelic acid
XXXGDGSGTIP

(19) United States
(12) Patent Application Publication
(10) Pub. No.: US 2025/0177537 A1
(43) Pub. Date: Jun. 5, 2025

(54) AN ANTIBIOTIC, METHOD OF PREPARATION AND METHOD OF TREATMENT THEREOF

(71) Applicant: National Taiwan University, Taipei (TW)

(72) Inventors: Chung-Han CHU, Taipei (TW); Shao-Lun CHIOU, Taipei (TW)

(21) Appl. No.: 18/965,907

(22) Filed: Dec. 2, 2024

Related U.S. Application Data
(60) Provisional application No. 63/605,584, filed on Dec. 3, 2023.

(51) Int. Cl. A61K 47/54; A61K 31/69; A61K 38/12; A61P 31/14; C07K 1/06

(52) U.S. Cl. CPC A61K 47/542; A61K 31/69; A61K 38/12; A61P 31/14; C07K 1/063

(57) ABSTRACT

The present invention provides an antibiotic derived from a calcium dependent antibiotic wherein the antibiotic of the present invention has a reduced or no calcium-dependency. In an embodiment, the antibiotic of the present invention is a boron dependent antibiotic. The present invention also provides method of preparation and method of treatment of the antibiotic of the present invention.

### INCORPORATION BY REFERENCE OF A SEQUENCE LISTING XML

A Sequence Listing is provided herewith as a Sequence Listing XML, “SequenceNTU1US.xml” created on Dec. 2, 2024 and having a size of 103 KB. The contents of the Sequence Listing XML are incorporated by reference herein in their entirety.

### FIELD OF THE INVENTION

The present invention provides an antibiotic compound with a reduced or no calcium-dependency, method of preparation, and method of using thereof.

### BACKGROUND OF THE INVENTION

The antibiotic resistance crisis poses a significant and imminent threat to humanity. [1] Infectious diseases are already one of the top causes of death worldwide and have been steadily on the rise. Today, one in every eight deaths globally result directly or indirectly from infectious diseases, taking a toll of approximately 7 million lives each year, of which 5 million are associated with drug-resistant microbial pathogens. [2] It was estimated that by 2050 antimicrobial resistance will cause 10 million deaths a year and cost the global economy a cumulative $100 trillion US dollars. [3] Effective new antibiotics for combating these recalcitrant microbial pathogens are desperately needed to avert this trend.

Calcium-dependent antibiotics (CDAs) are a family of nonribosomal peptide natural products that interact with a variety of organophosphate intermediates in the cell wall biosynthesis pathway to inhibit bacterial growth. [4] They present a rich reservoir of new drug candidates to combat antibiotic resistant pathogens for their diverse mechanisms of action (MOA). For example, malacidin binds to lipid II and is highly effective in a mouse model in sterilizing wounds infected by methicillin-resistant Staphylococcus aureus (MRSA). [5] Friulimicin B binds to undecaprenyl phosphate (C55P) and was a promising drug candidate until its Phase I clinical trial was terminated due to unfavorable pharmacokinetics. [6] Daptomycin forms a tripartite complex with phosphatidylglycerol and bactoprenol-containing cell envelope precursors, leading to altered membrane curvature and cell death. [7] It was approved for clinical use in 2003, typically as a second-line Gram-positive antibiotic for the treatment of MRSA and vancomycin-resistant Enterococci (VRE) infections. As the name suggests, CDAs are active only in the presence of calcium cations (Ca²⁺, henceforth referred to simply as calcium). However, free calcium in human extracellular matrix, which ranges from 1.1 to 1.4 mM, [8] is barely sufficient for the full activation of most CDAs. [5, 9] Therefore, there is a need to explore the molecular engineering of CDAs in hope of lowering their calcium dependence to potentially enable broader applications.

### SUMMARY OF THE INVENTION

An antibiotic of formula (I)

X—Y—Z Formula (I);
wherein:
X comprises a long-chain fatty acid;
Y comprises a linear peptide comprising one, two, or three amino acids;
Z comprises a cyclic peptide comprising nine or ten amino acids; and
wherein the amino acid sequence of the antibiotic is identical or similar to the amino acid sequence of the peptide of a parent calcium dependent antibiotic (CDA) except at least one aspartic acid (Asp) residue in the amino acid sequence of the peptide of the parent CDA is replaced with a non-Asp residue in the amino acid sequence of Y—Z of the CDA derivative, wherein the parent CDA comprises daptomycin, taromycin A, taromycin B, A54145, CDA, cadaside A, cadaside B, malacidin A, malacidin B, laspartomycin, amphomycin A, amphomycin B, amphomycin C, amphomycin D, friulimicin A, friulimicin B, friulimicin C, friulimicin D, or a synthetic analogue thereof comprising similar or improved calcium-dependent antibacterial activity.

A method of treatment of bacterial infection in a subject comprising the step of administration of a therapeutically effective amount of the antibiotic of Formula (I) to the subject.

### BRIEF DESCRIPTION OF THE DRAWINGS

FIG. 1 illustrates an embodiment of calcium replacement by PBA as the anchor moiety in the synthetic LspC analogs. FIG. 1A is a cartoon illustration of calcium induced conformational change of LspC and binding of C55P. FIG. 1B illustrates a structure of LspC in complex with calcium and C10P (PDB ID: 5O0Z). [9b] FIG. 1C illustrates a structural model based on MD simulation of the synthetic LspC analog B1. Structures in FIGS. 1B and 1C were visualized by PyMOL. The Asp/Ca2+ and Ser/phenylboronic acid (PBA) interactions in LspC and B1 are colored coded as follows-carbon in green, nitrogen in blue, oxygen in red, phosphorous in purple, boron in yellow, and calcium in pink; the rest of the peptides are shown in light grey. The hydrocarbon portion of C10P and PBA are shown in black. FIG. 1D illustrates the chemical equilibrium between boronic acid, boronic ester, and boronate ester.

FIG. 2 illustrates various embodiments of synthetic LspC analogs. FIG. 2A illustrates the general structure of LspC and its synthetic analogs. Residues in light blue shade constitute the Asp-X-Asp-Gly (DXDG) motif that is regarded as the defining feature of a CDA. The position of the PBA anchor relative to the rest of the peptide can be fine-tuned by replacing Asp1 and Asp7 by Ser or Hse, which differ by only one methylene (—CH2-) unit. FIG. 2B shows that S1 is identical to LspC except for minor differences in their fatty acyl chain. We designed four synthetic LspC analogs (B1-B4) to cover all combinations of Ser/Hse substitution at residue land 7 (red). Amino acids are shown in three letter codes, wherein capitalized and lowercase codes denote L- and D-form residues, respectively. Aside from D-amino acids, LspC contains two amino acids with noncanonical side-chains, i.e., 2,3-diaminopropionic acid (Dap) and D-pipecolic acid (pip).

FIG. 3 illustrates the MICs of LspC and its synthetic analogs against Bacillus subtilis.

FIG. 4 illustrates the calcium dependence, PBA dependence, cytotoxicity, and C10P antagonism of the LspC analogs. FIG. 4A shows that the MICs of S1 and B1 were determined against B. subtilis in LB supplemented with the indicated amount of calcium (CaCl2); assays for the latter were also supplemented with PBA (50 μg/mL (0.41 mM)). The antibacterial activity of S1 was strongly dependent on calcium, whereas that of B1 was completely independent of calcium across all concentrations we tested. FIG. 4B is a MIC vs. PBA concentration plot showing that B1 can be activated by very small amounts of PBA, suggesting that the boronate ester formation in the B1/PBA complex is highly specific and extremely stable. FIG. 4C shows the MTT assay was used to assess the cytotoxicity of our compounds against HEK293T cells. The IC50s of B1 and PBA, alone or in combination, are nearly two orders of magnitude higher than what is needed to suppress bacterial growth. FIG. 4D shows the resulting MICs of B1, B3, and B4 tested with or without C10P supplement (˜10-fold molar excess relative to peptide) against S. aureus. The presence of C10P abolished the activity of all three synthetic LspC analogs, suggesting that C10P competed for binding and prevented them from sequestering the actual substrate C55P. “>” denotes no growth inhibition at the highest concentration tested (128 μg/mL (104 μM))

FIG. 5 illustrates the direct observation of various B1 complexes. FIG. 5A shows the TLC plate was developed using 2:1 (v/v) DCM/MeOH as the mobile phase and KMnO4 for staining. Mobility shifts upon the addition of C10P, and then calcium (lane 5 to 7), hinted at the successive formation of a ternary and a quaternary complex. FIGS. 5B and 5C show analysis results of B1 complex formation was monitored by using NESI-HRMS in both the negative (FIG. 5B) and positive (FIG. 5C) modes. Peaks corresponding to B1 and its binary, ternary, and quaternary complex were denoted by yellow, green, blue, and red circles. See FIGS. 15-18 and 24-25 for complete peak assignments. FIG. 5D shows the result of a series of 31P NMR experiments was used to monitor B1 complex formation. We began by acquiring a spectrum of C10P alone, followed by successive additions of PBA, B1, and calcium. The appearance of two peaks upon the addition of B1 suggests the formation of two slightly different B1/PBA/C10P ternary complexes. They exchange slowly but eventually do equilibrate, leaving the 80.0 complex as the only observable peak (FIG. 19-20).

FIG. 6 illustrates the activity spectrum of B1. ^{a,b}The denoted Enterococcus and Staphylococcus strains are vancomycin and methicillin resistant strains (VRE and MRSA), respectively. ^{c}“>” indicates no noticeable growth suppression at the highest concentration tested (128 μg/mL (106 μM)).

FIG. 7 illustrates the ¹H NMR spectrum of Fmoc-D-allo-Thr-OH.

FIG. 8 illustrates the ¹³C NMR spectrum of Fmoc-D-allo-Thr-OH.

FIG. 9 illustrates the HPLC and HRMS of S1. S1 Formula C₅₈H₉₄N₁₂O₁₉. [S1+H]+ calcd: 1263.6836, obsd: 1263.6776. [S1+Na]+ calcd: 1285.6656, obsd: 1285.6610. [S1+K]+ calcd: 1301.6395, obsd: 1301.6241.

FIG. 10 illustrates the HPLC and HRMS of B1. Formula C₅₆H₉₄N₁₂O₁₇. [B1+H]+ calcd: 1207.6938, obsd: 1207.6943. [B1+Na]+ calcd: 1229.6758, obsd: 1207.6798.

FIG. 11 illustrates the HPLC and HRMS of B2. Formula C₅₈H₉₈N₁₂O₁₇. [B2+H]+ calcd: 1235.7251, obsd: 1235.7131. [B2+Na]+ calcd: 1257.7071, obsd: 1257.6954.

FIG. 12 illustrates the HPLC and HRMS of B3. Formula C₅₈H₉₈N₁₂O₁₇. [B3+H]+ calcd: 1221.7095, obsd: 1221.7104. [B3+Na]+ calcd: 1243.6914, obsd: 1243.6953. [B3+K]+ calcd: 1259.6653, obsd: 1259.6570.

FIG. 13 illustrates the HPLC and HRMS of B4. Formula C₅₈H₉₈N₁₂O₁₇. [B4+H]+ calcd: 1221.7095, obsd: 1221.7131. [B4+Na]+ calcd: 1243.6914, obsd: 1243.6971. [B4+K]+ calcd: 1259.6653, obsd: 1259.6581.

FIG. 14 illustrates the molecular dynamic simulation of LspC analog B1. a Left: Chemical structure of the simulated B1-PBA-C10P complex. The carbon chain length of C10P and fatty acyl chain was drawn based on the resolved atoms from a co-crystal structure of LspC in complex with calcium and C10P (PDB ID: 5O0Z). Middle: A snapshot of the B1-PBA-C10P+Ca2+ complex before an amide flip between pip3 and Gly4. Right: A snapshot of the B1-PBA-C10P+Ca2+ complex after an amide flip between pip3 and Gly4. The flipped amide bond was circled in red. All hydrogen atoms except the one in the flipped amide bond were not shown for clarity. b Changes in (ϕ,ψ) backbone dihedral angles of each amino acid of B1 over 100 ns across five simulations. (ϕ,ψ) angles from the simulations were colored in red and blue, respectively. The (ϕ,ψ) angles in PDB ID: 5O0Z were shown in solid green line and dashed orange line, respectively, to compare the stability of the corresponding residues in the simulations.

FIG. 15 illustrates the B1 complexes detected by NESI-HRMS at pH 7 (negative mode).

FIG. 16 illustrates the B1 complexes detected by NESI-HRMS at pH 7 (positive mode).

FIG. 17 illustrates the B1 complexes detected by NESI-HRMS at pH 8 (negative mode).

FIG. 18 illustrates the B1 complexes detected by NESI-HRMS at pH 8 (positive mode).

FIG. 19 illustrates the ¹¹B NMR spectra of various B1 complexes. From top to bottom: B1/PBA/C10P/Ca²⁺; B1/PBA/C10P; PBA/C10P; PBA.

FIG. 20 illustrates the ³¹P NMR spectra of various B1 complexes. From top to bottom: B1/PBA/C10P/Ca²⁺ (one week after mixing); B1/PBA/C10P (immediately after mixing); B1/PBA/C10P; PBA/C10P; C10P alone.

FIG. 21 illustrates the comparison of known CDAs peptide sequence. Residues in bold type represent macrocyclization site; acidic and basic residues are in red and blue respectively; residues highlighted by orange are modified by high-electronegativity-atom containing groups (i.e. O, Cl); underlined residues are modified by methyl group; FA is acronym of fatty acid. Number of acidic (exocyclic part) and basic residues are summed, and the net charges at physiological pH value are also stated.

FIG. 22 illustrates the MICs of synthetic LspC peptides against Bacillus subtilis. The symbol “>” indicates no noticeable growth suppression at the highest concentration tested (128 μg/mL); “ND” stands for not determined; MICs of LspC are values reported in the literature.

FIG. 23 illustrates the B1 complexes see in NESI-HRMS (negative mode)—calculated vs. observed.

FIG. 24 illustrates the B1 complexes see in NESI-HRMS (positive mode)—calculated vs. observed.

FIG. 25 illustrates the Bacteria growth conditions. All media were supplemented with CaCl₂) (1.2 mM) and PBA (0.05 mg/mL) unless noted otherwise.

FIG. 26 illustrates the antibacterial activity of peptide (B1) in combination with various boron species tested by the growth inhibitory zone assay. B1 was mixed with various boron species at 50 mM each (final concentration). These mixtures were then spotted on a lawn of Bacillus subtilis. After 12 hours of incubation at room temperature, combinations effective at inhibiting bacterial growth would appear as a circular zone. Sixteen boron species (1-16) were tested and all were effective: (1) 4-hydroxylphenylboronic acid, (2) phenylboronic acid, (3) 3-aminophenylboronic acid, (4) 4-aminophenylboronic acid, (5) 4-cyanophenylboronic acid, (6) 4-methoxyphenylboronic acid, (7) 4-pyridylboronic acid, (8) 3-hydroxylphenylboronic acid, (9) 4-fluorophenylboronic acid, (10) 2-formylphenylboronic acid, (11) 4-(methylthio) phenylboronic acid, (12) 4-methoxycarbonyl phenylboronic acid, (13) 3-pyridylboronic acid, (14) 4-tolylboronic acid, (15) 3,5-dimethylphenylboronic acid, (16) 3,5-(bis(trifluoromethyl)phenyl) boronic acid.

### DETAILED DESCRIPTION OF THE INVENTION

As used in this specification and in claims which follow, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

As used herein, the term “about” as a modifier to a quantity is intended to mean ±20%, ±15%, ±10% or ±5% inclusive of the quantity being modified.

As used herein, the term “subject,” “individual” or “patient” is used interchangeably herein, which refers to a vertebrate, preferably a mammal, more preferably a human. Mammals include, but are not limited to, murines, simians, humans, farm animals, sport animals, and pets.

As used herein, the term “effective amount” or “a therapeutically effective amount” of a drug or pharmacologically active agent comprises administering an amount necessary to achieve a desired result. The exact amount required will vary from subject to subject, depending on the species, age, general condition of the subject, the severity of the disease, the particular active agent, its mode of administration, the desired outcome, and the like. In certain embodiments of the present invention, a “therapeutically effective amount” of a compound or pharmaceutical composition is that amount effective for inhibiting progression or reversing of any disease disclosed herein in a subject or a biological sample (e.g., in cells). In certain embodiments, disease progression is inhibited by about 10%, about 20%, about 30%, about 40%, about 50%, about 60%, about 70%, about 80%, about 90%, about 95%, or about 99%. In certain embodiments, the compound inhibits disease progression by at least about 25%, at least about 50%, at least about 75%, or at least about 90%. In certain embodiments of the present invention, a “therapeutically effective amount” refers to an amount of a composition sufficient to reversal of disease. In certain embodiments, the disease is reversed by about 1%, about 5%, about 10%, about 20%, about 30%, about 40%, about 50%, about 60%, about 70%, about 80%, about 90%, about 95%, about 99% or any numbers and number ranges falling within these values.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising CDRs and being capable of binding an antigen or influenza virus-infected cell.

An “amino acid sequence,” “peptide,” “polypeptide,” or “protein” is a sequence of amino acids (including both naturally occurring and non-naturally occurring amino acids), which may be found in native proteins or may be artificially engineered as recombinant proteins or parts thereof. The term should also be understood to include, as equivalents, polypeptides with additional modifications, including but not limited to phosphorylation, glycosylation, lipidation, etc.

As used herein, “sequence identity” and “% identity,” refers to the value determined by comparing two optimally aligned sequences over a comparison window, wherein a portion of the sequence in the comparison window may comprise additions or deletions as compared to the reference sequence for optimal alignment of the two sequences. The number of positions at which identical amino acid residues occur in both sequences is determined, yielding the number of matched positions, which is divided by the total number of positions in the window of comparison and the result multiplied by 100 to yield the percentage of sequence identity. The comparison window is the entire length of the sequence being referred to unless indicated otherwise.

As used herein, “% similarity” is calculated as described for “% identity,” with the exception that the hydrophobic residues Ala, Val, Phe, Pro, Leu, Ile, Trp, Met, and Cys are similar; the basic residues Lys, Arg, and His are similar; the acidic residues Glu and Asp are similar; and the hydrophilic, uncharged residues Gln, Asn, Ser, Thr, and Tyr are similar. The remaining natural amino acid Gly is not similar to any other amino acid in this context.

As used herein, the term “protecting group” refers to any chemical compound that may be used to prevent a group on a molecule from undergoing a chemical reaction while chemical change occurs elsewhere in the molecule. Groups that may need protecting include hydroxyl, amino, carboxylic acids and carboxyamino groups. Numerous protecting groups are known to those skilled in the art and examples can be found in “Protective Groups in Organic Synthesis” by Theodora W. Greene and Peter G. M. Wuts, John Wiley and Sons, New York, 3rd Edition 1999, hereafter Greene.

As used herein, the term “amino protecting group” refers to any chemical compound that may be used to prevent an amino group on a molecule from undergoing a chemical reaction while chemical change occurs elsewhere in the molecule. Numerous amino protecting groups are known to those skilled in the art and examples can be found in Greene. Examples of “amino protecting groups” include phthalimido, trichloroacetyl, STA-base, benzyloxycarbonyl, t-butoxycarbonyl, t-amyloxycarbonyl, isobornyloxycarbonyl, adamantyloxycarbonyl, chlorobenzyloxycarbonyl, nitrobenzyloxycarbonyl or the like. Preferred amino protecting groups are “carbamate amino protecting groups” which are defined as an amino protecting group that when bound to an amino group forms a carbamate, or the azido group. Preferred amino carbamate protecting groups are allyloxycarbonyl (alloc), carbobenzyloxy (CBZ), 9-fluorenylmethoxycarbonyl (Fmoc) and tert-butoxycarbonyl protecting groups.

As used herein, the term “proteinogenic amino acid” refers to amino acids that are incorporated into proteins during the translation in an organism. Examples of proteinogenic amino acids include L-aspartic acid, L-lysine, L-arginine, L-histidine, L-glutamic acid, L-tyrosine, L-threonine, L-glutamine, L-asparagine, L-cysteine, L-serine, L-proline, L-phenylalanine, L-tryptophan, L-methionine, L-isoleucine, glycine, L-leucine, L-valine, and L-alanine, which are genetically encoded amino acids in the standard genetic code. L-selenocysteine and L-pyrrolysine are proteinogenic amino acids not encoded by the standard genetic code but can be incorporated into proteins by different translation mechanism. In an embodiment, a proteinogenic amino acid “Xaa” is used interchangeably with “L-Xaa”. On the other hand, the term “non-proteinogenic amino acid” refers amino acids that cannot be incorporated into proteins during the translation in an organism. In an embodiment, non-proteinogenic amino acid comprises post-translationally modified proteinogenic amino acid. Examples of non-proteinogenic amino acids include D-amino acids, ornithine (Orn), kynurenine (Kyn), 4-hydroxyphenylglycine (Hpg), diaminopimelic acid (Dap), pipecolic acid (Pip), 3-methyl-glutamic acid, 6-chloro-tryptophan, 4-chloro-kynurenine, 3-hydroxyl-L-asparagine, sarcosine, 3-methoxy-aspartic acid, beta-hydroxy-asparagine, Z-2,3-dehydrotryptophan, beta-hydroxy-aspartic acid, gamma-hydroxy-glutamic acid, 3-methyl-aspartic acid, 4-methyl-proline, dimainobutyric acid (Dab), any modified variants thereof, and many others.

The present invention provides an antibiotic having structure of formula (I):

X—Y—Z Formula (I);
wherein X comprises a long-chain fatty acid, Y comprises a linear peptide comprising at least one amino acid, and Z comprises a cyclic peptide comprising at least six amino acids.

In an embodiment, the antibiotic compound of the present invention is derived from a parent calcium dependent antibiotic wherein the parent calcium dependent antibiotic comprises daptomycin, taromycin A, taromycin B, A54145, CDA, cadaside A, cadaside B, malacidin A, malacidin B, laspartomycin, amphomycin A, amphomycin B, amphomycin C, amphomycin D, friulimicin A, friulimicin B, friulimicin C, or friulimicin D, including synthetic analogue thereof comprising similar or improved calcium-dependent antibacterial activity.

In an embodiment, X comprises a fatty acid chain identical to the fatty acid chain of any of the parent calcium dependent antibiotic. In an embodiment, X comprises a fatty acid chain with a hydrophobicity about 80%, about 85%, about 90%, about 95%, about 100%, about 105%, about 110%, about 115%, about 120%, or any percentages or percentage ranges falling within these values as the hydrophobicity of fatty acid chain of any of the parent CDA. In an embodiment, X comprises a fatty acid chain comprising a carbon number of about 80%, about 85%, about 90%, about 95%, about 100%, about 105%, about 110%, about 115%, about 120%, or any percentages or percentage ranges falling within these values as the carbon number of fatty acid chain of any of the parent CDA. In an embodiment, X comprises a fatty acid chain with a hydrophobicity about 80%, about 85%, about 90%, about 95%, about 100%, about 105%, about 110%, about 115%, about 120%, or any percentages or percentage ranges falling within these values as the hydrophobicity of the fatty acid chain comprising —(CO)(CH₂)₁₄(CH₃), —(CO)(CH)₂(CH₂)₉(CH)(CH₃)₂, —(CO)(CH₂)₈(CH₃), —(CO)(CH)₄(CH₂)₂(CH₃), —(CO)(CH)₄(CH)(CH₃)(CH₂)(CH₃), —(CO)(CH₂)₆(CH)(CH₃)₂, —(CO)(CH₂)₆(CH)(CH₃)(CH₂)(CH₃), —(CO)(CHOCH)(CH₂)₂(CH₃), —(CO)(CH)₄(CH₂)₂(CH)(CH₃)₂, —(CO)(CH)₄(CH₂)₂(CH)(CH₃)(CH₂)(CH₃), —(CO)(CH₂)(CH)₂(CH₂)₆(CH)(CH₃)₂, —(CO)(CH₂)(CH)₂(CH₂)₇(CH)(CH₃)₂, —(CO)(CH₂)(CH)₂(CH₂)₅(CH)(CH₃)(CH₂)(CH₃), or —(CO)(CH₂)(CH)₂(CH₂)₇(CH)(CH₃)(CH₂)(CH₃).

In an embodiment, Y comprises at least one amino acid such as one, two, three, four, five, or six amino acids. In an embodiment, Y does not comprise an Asp residue or a variant thereof such as but not limited to D-Asp, 3-methoxy-aspartic acid, beta-hydroxy-aspartic acid, or 3-methyl-aspartic acid. In an embodiment, Y comprises proteinogenic amino acid, non-proteinogenic amino acid, or a combination thereof. In an embodiment, Y comprises Trp-Asn-Xaa, Trp-Glu-Asn, Ser, Ala-Glu-Tyr, Xaa, or Asn, wherein each Trp comprises Trp or 6-chloro-tryptophan, wherein each Asn comprises Asn, D-Asn, or 3-hydroxyl-L-asparagine, wherein each Glu comprises D-Glu, and wherein each Xaa can be any amino acid. In an embodiment, Xaa comprises Asp or a variant thereof such as but not limited to D-Asp, 3-methoxy-aspartic acid, beta-hydroxy-aspartic acid, or 3-methyl-aspartic acid. In another embodiment, Xaa comprises a non-Asp residue such as but not limited to lysine, arginine, histidine, glutamic acid, tyrosine, threonine (Thr), homothreonine (Hth), glutamine, asparagine, cysteine, serine (Ser), homoserine (Hse), proline, phenylalanine, tryptophan, methionine, isoleucine, glycine, leucine, valine, or alanine. In an embodiment, Y does not comprise an amino acid that interacts with a calcium ion when the antibiotic of the present invention is in contact with a calcium ion. In an embodiment, the interaction of the antibiotic of the present invention with calcium ion increases when at least one of the non-Asp residues of Y is replaced with an Asp residue or a variant thereof such as but not limited to D-Asp, 3-methoxy-aspartic acid, beta-hydroxy-aspartic acid, or 3-methyl-aspartic acid. In an embodiment, Y comprises a Ser or a Hse.

In an embodiment, Z comprises at least six amino acids such as six, seven, eight, nine, ten, eleven, twelve, thirteen, fourteen, fifteen, sixteen, seventeen, or eighteen amino acids. In an embodiment, Z does not comprise an Asp residue or a variant thereof such as but not limited to D-Asp, 3-methoxy-aspartic acid, beta-hydroxy-aspartic acid, or 3-methyl-aspartic acid. In an embodiment, Z comprises one, two, or three Asp residues or a variant thereof such as but not limited to D-Asp, 3-methoxy-aspartic acid, beta-hydroxy-aspartic acid, or 3-methyl-aspartic acid. In an embodiment, Z comprises proteinogenic amino acid, non-proteinogenic amino acid, or a combination thereof. In an embodiment, the amino acid sequence of Z is identical or similar to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

SEQ ID NO 1:
Thr-Gly-Orn-Xaa-Ala-Xaa-Gly-Ser-Glu-Kyn,
SEQ ID NO 2:
Thr-Gly-Orn-Xaa-Ala-Xaa-Gly-Ala-Glu-Kyn,
SEQ ID NO 3:
Thr-Gly-Ala-Xaa-Lys-Xaa-Gly-Asn-Glu-Ile,
SEQ ID NO 4:
Thr-Trp-Xaa-Xaa-Hpg-Xaa-Gly-Asn-Glu-Trp,
SEQ ID NO 5:
Thr-Ile-Xaa-Xaa-Pro-Gly-Glu-Glu-Gly,
SEQ ID NO 6:
Dap-Val-Lys-Xaa-Xaa-Gly-Xaa-Val-Pro,
SEQ ID NO 7:
Dap-Pip-Gly-Xaa-Gly-Xaa-Gly-Thr-Ile-Pro,
SEQ ID NO 8:
Dap-Pip-Xaa-Xaa-Gly-Xaa-Gly-Dab-Val-Pro,

wherein the two underlined amino acids of each sequences from SEQ ID NO: 1-8 are linked to form a cyclic peptide, wherein the amino acid residue highlighted italic comprises a D-amino acid, wherein Glu highlighted bold comprises Glu, 3-methyl-glutamic acid, or gamma-hydroxy-glutamic acid, wherein Kyn highlighted bold comprises 4-chloro-kynurenine, wherein Gly highlighted bold comprises sarcosine, wherein Ile highlighted bold comprises Ile or Val, wherein Asn highlighted bold comprises beta-hydroxy-asparagine or a phosphorylated form thereof, wherein Trp highlighted bold comprises Trp or Z-2,3-dehydrotryptophan, wherein Pro highlighted bold comprises 4-methyl-proline, wherein each Xaa can be any amino acid. In an embodiment, Xaa comprises Asp or a variant thereof such as but not limited to D-Asp, 3-methoxy-aspartic acid, beta-hydroxy-aspartic acid, or 3-methyl-aspartic acid. In an embodiment, Xaa comprises a non-Asp residue such as but not limited to lysine, arginine, histidine, glutamic acid, tyrosine, threonine (Thr), homothreonine (Hth), glutamine, asparagine, cysteine, serine (Ser), homoserine (Hse), proline, phenylalanine, tryptophan, methionine, isoleucine, glycine, leucine, valine, or alanine. In an embodiment, the amino acid sequence of Z is identical to SEQ ID NO: 9, SEQ ID NO: 10, SEQ ID NO: 11, SEQ ID NO: 12, SEQ ID NO: 13, or SEQ ID NO: 14.

SEQ ID NO: 9:
Thr-Gly-Orn-Asp-Ala-Xaa-Gly-Ser-Glu-Kyn,
SEQ ID NO: 10:
Thr-Gly-Orn-Asp-Ala-Xaa-Gly-Ala-Glu-Kyn,
SEQ ID NO: 11:
Thr-Gly-Ala-Asp-Lys-Xaa-Gly-Asn-Glu-Ile,
SEQ ID NO: 12:
Thr-Trp-Xaa-Asp-Hpg-Xaa-Gly-Asn-Glu-Trp
SEQ ID NO: 13:
Dap-Pip-Gly-Asp-Gly-Xaa-Gly-Thr-Ile-Pro,
SEQ ID NO: 14:
Dap-Pip-Xaa-Asp-Gly-Xaa-Gly-Dab-Val-Pro,

wherein the two underlined amino acids of each sequences from SEQ ID NO: 9-14 are linked to form a cyclic peptide, wherein the amino acid residue highlighted italic comprises a D-amino acid, wherein Glu highlighted bold comprises Glu, 3-methyl-glutamic acid, or gamma-hydroxy-glutamic acid, wherein Kyn highlighted bold comprises 4-chloro-kynurenine, wherein Gly highlighted bold comprises sarcosine, wherein Ile highlighted bold comprises Ile or Val, wherein Asn highlighted bold comprises beta-hydroxy-asparagine or a phosphorylated form thereof, wherein Trp highlighted bold comprises Trp or Z-2,3-dehydrotryptophan, wherein each Xaa can be any amino acid. In an embodiment, Xaa comprises Asp or a variant thereof such as but not limited to D-Asp, 3-methoxy-aspartic acid, beta-hydroxy-aspartic acid, or 3-methyl-aspartic acid. In an embodiment, Xaa comprises a non-Asp residue such as but not limited to lysine, arginine, histidine, glutamic acid, tyrosine, threonine (Thr), homothreonine (Hth), glutamine, asparagine, cysteine, serine (Ser), homoserine (Hse), proline, phenylalanine, tryptophan, methionine, isoleucine, glycine, leucine, valine, or lanine. In an embodiment, Z does not comprise an amino acid capable of interacting with a calcium ion. In an embodiment, the interaction of any embodiment of the antibiotic of formula (I) of the present invention with calcium ion increases when at least one of the non-Asp residues of Z is replaced with an Asp residue or a variant thereof such as but not limited to D-Asp, 3-methoxy-aspartic acid, beta-hydroxy-aspartic acid, or 3-methyl-aspartic acid. In an embodiment, amino acid sequence of Z is at least about 80%, about 85%, about 90%, about 95%, or about 100% identical or similar to SEQ ID NO: 15 or SEQ ID NO: 16.

SEQ ID NO: 15:
Dap-Pip-Gly-Asp-Gly-Ser-Gly-Thr-Ile-Pro,
SEQ ID NO: 16:
Dap-Pip-Gly-Asp-Gly-Hse-Gly-Thr-Ile-Pro,

wherein the two underlined amino acids of each sequences from SEQ ID NO: 15-16 are linked to form a cyclic peptide, wherein the amino acid residue highlighted italic comprises a D-amino acid.

In an embodiment, amino acid sequence of Y—Z is at least about 80%, about 85%, about 90%, about 95%, or about 100% identical or similar to SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29.

SEQ ID NO: 17 (daptomycin):
Trp-Asn-Xaa-Thr-Gly-Orn-Xaa-Ala-Xaa-Gly-Ser-Glu-
Kyn,
SEQ ID NO: 18 (taromycin):
Trp-Asn-Xaa-Thr-Gly-Orn-Xaa-Ala-Xaa-Gly-Ala-Glu-
Kyn,
SEQ ID NO: 19 (A54145):
Trp-Glu-Asn-Thr-Gly-Ala-Xaa-Lys-Xaa-Gly-Asn-Glu-
Ile,
SEQ ID NO: 20 (CDA):
Ser-Thr-Trp-Xaa-Xaa-Hpg-Xaa-Gly-Asn-Glu-Trp,
SEQ ID NO: 21 (cadaside):
Ala-Glu-Tyr-Thr-Ile-Xaa-Xaa-Pro-Gly-Glu-Glu-Gly,
SEQ ID NO: 22 (malacidin):
Xaa-Dap-Val-Lys-Xaa-Xaa-Gly-Xaa-Val-Pro,
SEQ ID NO: 23 (laspartomycin):
Xaa-Dap-Pip-Gly-Xaa-Gly-Xaa-Gly-Thr-Ile-Pro,
SEQ ID NO: 24 (amphomycin):
Xaa-Dap-Pip-Xaa-Xaa-Gly-Xaa-Gly-Dab-Val-Pro,
SEQ ID NO: 25 (friulimicin):
Asn-Dap-Pip-Xaa-Xaa-Gly-Xaa-Gly-Dab-Val-Pro,
SEQ ID NO: 26 (B1):
Ser-Dap-Pip-Gly-Asp-Gly-Ser-Gly-Thr-Ile-Pro,
SEQ ID NO: 27 (B2):
Hse-Dap-Pip-Gly-Asp-Gly-Hse-Gly-Thr-Ile-Pro,
SEQ ID NO: 28 (B3):
Ser-Dap-Pip-Gly-Asp-Gly-Hse-Gly-Thr-Ile-Pro,
SEQ ID NO: 29 (B4):
Hse-Dap-Pip-Gly-Asp-Gly-Ser-Gly-Thr-Ile-Pro,

wherein the two underlined amino acids of each sequences from SEQ ID NO: 17-29 are linked to form a cyclic peptide, wherein the amino acid residue highlighted italic comprises a D-amino acid, wherein Glu highlighted bold comprises Glu, 3-methyl-glutamic acid, or gamma-hydroxy-glutamic acid, wherein Kyn highlighted bold comprises 4-chloro-kynurenine, wherein Gly highlighted bold comprises sarcosine, wherein Ile highlighted bold comprises Ile or Val, wherein Asn highlighted bold comprises beta-hydroxy-asparagine or a phosphorylated form thereof, wherein Trp highlighted bold comprises Trp or Z-2,3-dehydrotryptophan, wherein Pro highlighted bold comprises 4-methyl-proline, wherein each Xaa can be any amino acid. In an embodiment, Xaa comprises Asp or a variant thereof such as but not limited to D-Asp, 3-methoxy-aspartic acid, beta-hydroxy-aspartic acid, or 3-methyl-aspartic acid. In an embodiment, Xaa comprises a non-Asp residue such as but not limited to lysine, arginine, histidine, glutamic acid, tyrosine, threonine (Thr), homothreonine (Hth), glutamine, asparagine, cysteine, serine (Ser), homoserine (Hse), proline, phenylalanine, tryptophan, methionine, isoleucine, glycine, leucine, valine, or alanine.

In an embodiment, the antibiotic of formula (I) of the present invention has a chemical structure identical to a parent CDA except at least one of the Asp residues or a variant thereof such as but not limited to D-Asp, 3-methoxy-aspartic acid, beta-hydroxy-aspartic acid, or 3-methyl-aspartic acid in the parent CDA is replaced with a non-Asp residue. In an embodiment the parent CDA comprises any prior art CDA. In an embodiment, the parent CDA comprises but not limited to daptomycin, taromycin A, taromycin B, A54145, CDA, cadaside A, cadaside B, malacidin A, malacidin B, laspartomycin, amphomycin A, amphomycin B, amphomycin C, amphomycin D, friulimicin A, friulimicin B, friulimicin C, or friulimicin D, or a synthetic analogue thereof comprising similar or improved calcium-dependent antibacterial activity. In an embodiment, the non-Asp residue comprises lysine, arginine, histidine, glutamic acid, tyrosine, threonine (Thr), homothreonine (Hth), glutamine, asparagine, cysteine, serine (Ser), homoserine (Hse), proline, phenylalanine, tryptophan, methionine, isoleucine, glycine, leucine, valine, or alanine. In an embodiment, the Asp residue of the peptide of the parent CDA replaced with a non-Asp residue is the first, second, third, fourth, fifth, sixth, seventh, eighth, nineth, tenth, eleventh or twelfth residue of the peptide of the parent CDA. In an embodiment, the non-Asp residue replacing the Asp residue of the parent CDA comprises Ser, Hse, Thr, or Hth.

In an embodiment in which the parent CDA comprises at least two Asp residues or a variant thereof such as but not limited to D-Asp, 3-methoxy-aspartic acid, beta-hydroxy-aspartic acid, or 3-methyl-aspartic acid, the antibiotic of formula (I) of the present invention is identical to the parent CDA except the first Asp residue or a variant thereof such as but not limited to D-Asp, 3-methoxy-aspartic acid, beta-hydroxy-aspartic acid, or 3-methyl-aspartic acid, the second Asp residue or a variant thereof such as but not limited to D-Asp, 3-methoxy-aspartic acid, beta-hydroxy-aspartic acid, or 3-methyl-aspartic acid, or a combination thereof of the peptide of the parent CDA are each independently replaced with Ser, Hse, Thr, or Hth. In an embodiment, the first Asp residue or a variant thereof such as but not limited to D-Asp, 3-methoxy-aspartic acid, beta-hydroxy-aspartic acid, or 3-methyl-aspartic acid of the peptide of the parent CDA is the first, second, third, fourth, fifth, sixth, seventh, eighth, nineth, tenth, eleventh or twelfth residue of the peptide of the parent CDA. In an embodiment, the second Asp residue or a variant thereof such as but not limited to D-Asp, 3-methoxy-aspartic acid, beta-hydroxy-aspartic acid, or 3-methyl-aspartic acid of the peptide of the parent CDA is the second, third, fourth, fifth, sixth, seventh, eighth, nineth, tenth, eleventh, twelfth, or thirteenth residue of the peptide of the parent CDA.

In an embodiment in which the parent CDA comprises LspC, the amino acid sequence of the antibiotic of formula (I) of the present invention is identical to the amino acid sequence of the parent CDA except the first Asp and the third Asp residues are both replaced with Ser. In an embodiment in which the parent CDA comprises LspC, the amino acid sequence of the antibiotic of formula (I) of the present invention is identical to the amino acid sequence of the parent CDA except the first Asp and the third Asp residues are both replaced with Hse. In an embodiment in which the parent CDA comprises LspC, the amino acid sequence of the antibiotic of formula (I) of the present invention is identical to the amino acid sequence of the parent CDA except the first Asp residue is replaced with Hse and the third Asp residue is replaced with Ser. In an embodiment in which the parent CDA comprises LspC, the amino acid sequence of the antibiotic of formula (I) of the present invention is identical to the amino acid sequence of the parent CDA except the first Asp residue is replaced with Ser and the third Asp residue is replaced with Hse. In an embodiment, the amino acid sequence of the antibiotic of formula (I) of the present invention is at least about 80%, about 85%, about 90%, about 95%, or about 100% identical or similar to SEQ ID NO: 26, SEQ ID NO: 27, SEQ ID NO: 28, or SEQ ID NO: 29.

In an embodiment, the replacement of one or more Asp residues or a variant thereof such as but not limited to D-Asp, 3-methoxy-aspartic acid, beta-hydroxy-aspartic acid, or 3-methyl-aspartic acid of a parent CDA with a non-Asp residue results in reduced calcium dependency for antibiotic activation. In an embodiment, the replacement of one or more Asp residues or a variant thereof such as but not limited to D-Asp, 3-methoxy-aspartic acid, beta-hydroxy-aspartic acid, or 3-methyl-aspartic acid of a parent CDA with a non-Asp residue results in reduced calcium dependency as well as boron dependency for activation of its antibacterial activity. In an embodiment, the replacement of one or more Asp residues or a variant thereof such as but not limited to D-Asp, 3-methoxy-aspartic acid, beta-hydroxy-aspartic acid, or 3-methyl-aspartic acid of a parent CDA with a non-Asp residue results in eliminated calcium dependency as well as boron dependency for activation of its antibacterial activity. In an embodiment, each of the one or more replaced Asp residue or a variant thereof such as but not limited to D-Asp, 3-methoxy-aspartic acid, beta-hydroxy-aspartic acid, or 3-methyl-aspartic acid is replaced with Ser, Hse, Thr, or Hth.

In an embodiment, the antibiotic of formula (I) of the present invention has a reduced calcium dependency for the activation of its antibacterial activity compared to the parent CDA from which it is derived by about 10%, about 20%, about 30%, about 40%, about 50%, about 60%, about 70%, about 80%, about 90% or about 100% including any percentages or percentage ranges falling within these values. In an embodiment, the antibacterial activity of the antibiotic of formula (I) of the present invention is dependent on a calcium ion and a non-calcium ion. In an embodiment, the antibacterial activity of the antibiotic of formula (I) of the present invention is dependent on a non-calcium ion. In an embodiment, the non-calcium ion comprises a boron ion.

In an embodiment, the antibiotic of formula (I) of the present invention comprises the following structures I-a, I-b, I-c, I-d, I-e, I-f, or I-g:

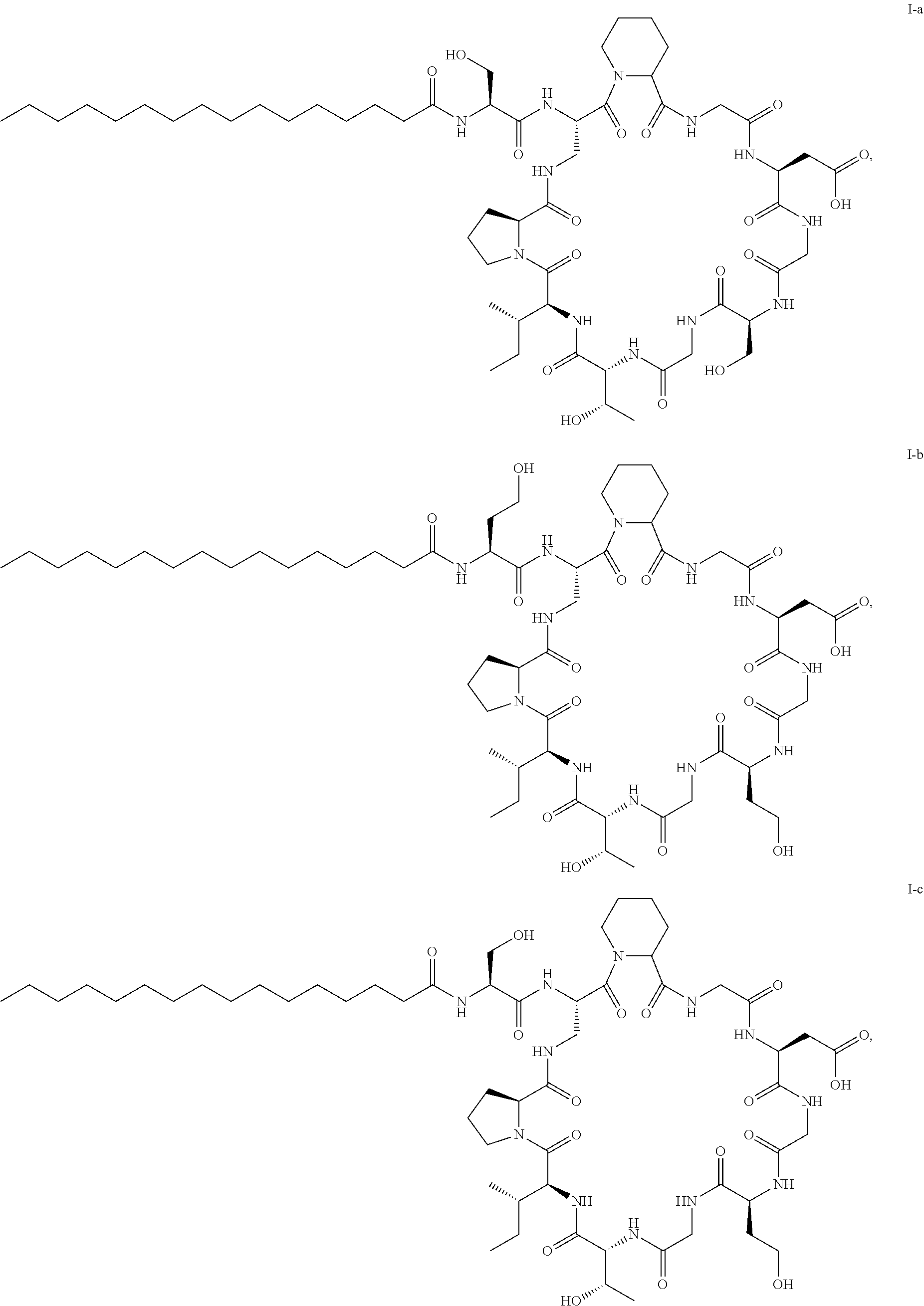

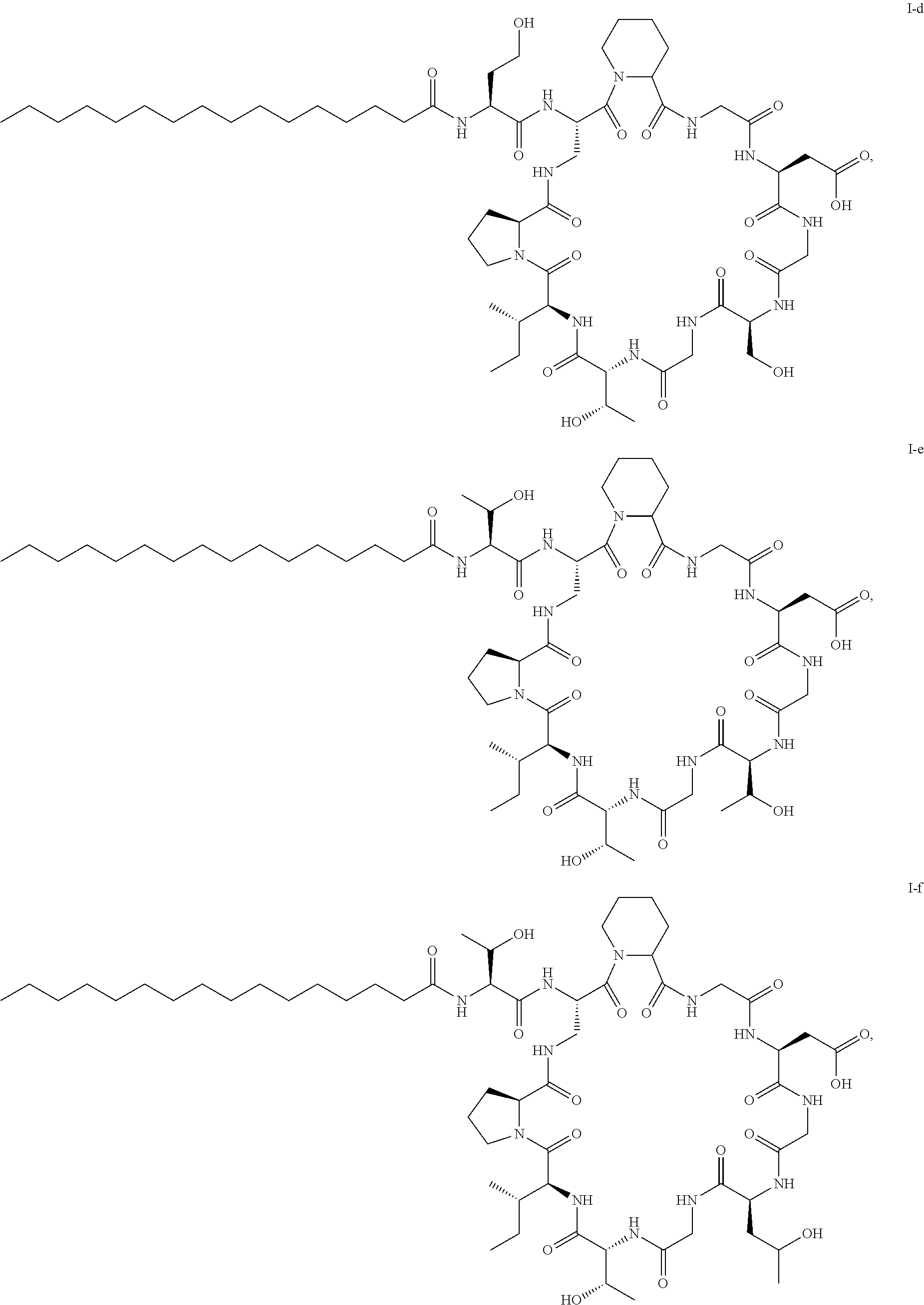

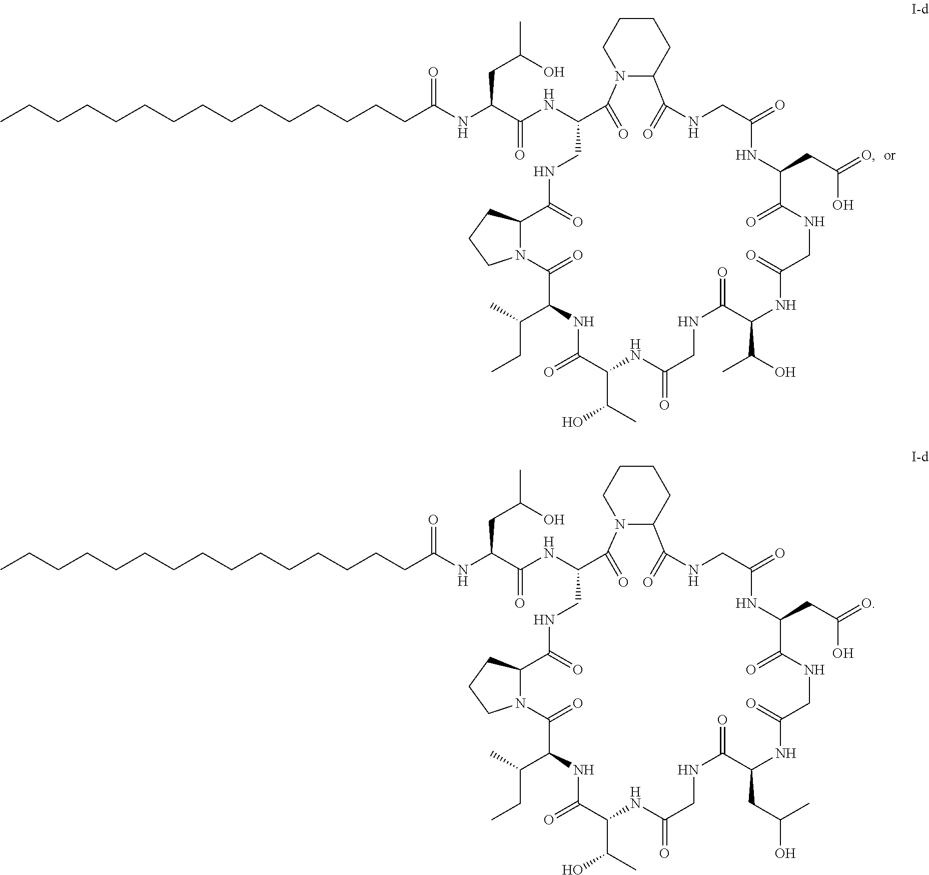 [SMILES: CCCCCCCCCCCCCCCC(=O)N[C@@H](CC(C)O)C(=O)N[C@H]1CNC(=O)[C@@H]2CCCN2C(=O)[C@H]([C@@H](C)CC)NC(=O)[C@@H]([C@H](C)O)NC(=O)CNC(=O)[C@H](C(C)O)NC(=O)CNC(=O)[C@H](CC(=O)O)NC(=O)CNC(=O)C2CCCCN2C1=O ; CCCCCCCCCCCCCCCC(=O)N[C@@H](CC(C)O)C(=O)N[C@H]1CNC(=O)[C@@H]2CCCN2C(=O)[C@H]([C@@H](C)CC)NC(=O)[C@@H]([C@H](C)O)NC(=O)CNC(=O)[C@H](CC(C)O)NC(=O)CNC(=O)[C@H](CC(=O)O)NC(=O)CNC(=O)C2CCCCN2C1=O]

The present invention also provides an antibiotic composition comprising any embodiment of the antibiotic of formula (I) of the present invention and an atom comprising a boron atom. In an embodiment, the antibiotic composition of the present invention further comprises a calcium atom. In an embodiment, the boron atom is in the form comprising a boronic acid, boronate, boronic ester, boronate ester. In an embodiment, boronic acid comprises phenylboronic acid (PBA), 4-hydroxylphenylboronic acid, 3-aminophenylboronic acid, 4-aminophenylboronic acid, 4-cyanophenylboronic acid, 4-methoxyphenylboronic acid, 4-pyridylboronic acid, 3-hydroxylphenylboronic acid, 2-formylphenylboronic acid, 4-(methylthio) phenylboronic acid, 4-methoxycarbonyl phenylboronic acid, 3-pyridylboronic acid, 4-tolylboronic acid, 3,5-dimethylphenylboronic acid, 3,5-(bis(trifluoromethyl)phenylf) boronic acid, 4-fluorophenylboronic acid, or a combination thereof. In an embodiment, the antibiotic of formula (I) of the present invention is capable of interacting with the boron atom in a biocompatible solution such as a physiological solution, growth media used in culturing bacteria, or growth media used in culturing eukaryotic cells in vitro. In an embodiment, the antibiotic of formula (I) of the present invention is capable of interacting with the boron atom and the calcium atom in a biocompatible solution. In an embodiment, the antibiotic of formula (I) of the present invention is not capable of interacting with a calcium atom in a biocompatible solution. In an embodiment, the boron atom comprises a boron ion. In an embodiment, the calcium atom comprises a calcium ion.

The present invention further provides a method of preparation of the antibiotic of formula (I) of the present invention. In an embodiment, the method of preparation of the antibiotic of formula (I) of the present invention comprises the step of performing solid-phase peptide synthesis (SPPS). In an embodiment, the SPPS step is performed using amino acid building blocks compatible with the fluorenylmethoxycarbonyl (Fmoc) chemistry or the tert-butoxycarbonyl (BOC) chemistry. In an embodiment, the SPPS step is performed using amino acid building blocks compatible with the Fmoc chemistry such as but not limited to amino acids comprising a N-terminal Fmoc protecting group and/or a side-chain protecting group not sensitive to a base such as but not limited to a tert-butyl protecting group, benzyl protecting group, trityl protecting group, 2-chlorotrityl protecting group, carboxybenzyl protecting group, allyl protecting group, allyloxycarbonyl protecting group, etc. In an embodiment, the method of preparation of the antibiotic of formula (I) of the present invention comprises the step of:
a [symbol] loading a first amino acid building block on a resin;
b [symbol] attaching a first peptide to the first amino acid building block loaded on the resin of step a;
c [symbol] attaching a long-chain fatty acid to the first peptide linked to the first amino acid building block loaded to the resin of step b;
d [symbol] attaching a second peptide to the first peptide at an amino acid residue of the first peptide to form a linear form of the CDA derivative of the present invention;
e [symbol] removing the linear form CDA derivative of step d from the resin;
f [symbol] circularizing the linear form CDA derivative of step e to form a crude CDA derivative of the present invention;
g [symbol] removing the protecting groups on the amino acid building blocks of the crude CDA derivative of step f to obtain the CDA derivative of the present invention.

In an embodiment, the first amino acid building block being loaded onto the resin comprises Asp or a variant thereof such as but not limited to D-Asp, 3-methoxy-aspartic acid, beta-hydroxy-aspartic acid, or 3-methyl-aspartic acid. In an embodiment, the first amino acid building block being loaded onto the resin comprises an amino acid residue that is the first, second, third, fourth, fifth, sixth, seventh, eighth, ninth, tenth, eleventh, twelfth, or thirteenth amino acid of the modified peptide of the CDA derivative. In an embodiment, the first amino acid building block being loaded onto the resin comprises an amino acid residue that is the fifth amino acid of the modified peptide of the CDA derivative. In an embodiment, the resin comprises a 2-chlorotrityl chloride (CTC) resin, Wang resin, or a Rink amide resin.

In an embodiment, the first peptide comprises at least four amino acids such as four, five, or six amino acids. In an embodiment, the first peptide comprises the first, second, third, fourth, fifth, sixth amino acid of the antibiotic of formula (I) of the present invention, or a combination thereof. In an embodiment, the first peptide comprises the first, second, third, and fourth amino acids of the modified peptide of the antibiotic of formula (I) of the present invention.

In an embodiment, attaching the first peptide to the first amino acid building block comprises the step of attaching one amino acid building block at a time to the amino acid or peptide linked to the resin according to the amino acid sequence of the chemical structure of the antibiotic of formula (I) of the present invention obtained after the chemical structure optimization step. For example, a second amino acid building block is attached to the first amino acid building block being loaded onto the resin, and a third amino acid building block is attached to the second amino acid building block linked to the first amino acid building block. In an embodiment, the amino acid building blocks are attached one by one according to the amino acid sequence of the chemical structure of the antibiotic of formula (I) of the present invention in a C-terminal to N-terminal direction. For example, the second amino acid building block attached to the first amino acid building block loaded onto the resin comprises the fourth amino acid residue of the antibiotic of formula (I) of the present invention, and the third amino acid building block attached to the second amino acid building block comprises the third amino acid residue of the antibiotic of formula (I) of the present invention.

In an embodiment, the step of attaching one amino acid building block at a time to the amino acid or peptide linked to the resin comprises a deblocking step and a coupling step. In an embodiment, the deblocking step comprises the addition of deblocking solution such as but not limited to piperidine solution to the amino acid or peptide linked to the resin. In an embodiment, the piperidine solution is supplemented with hydroxybenzotriazole (HOBt). In an embodiment, the deblocking step comprises the addition of palladium catalyst and phenylsilane in dichloromethane, the addition of triisopropylsilane in trifluoroacetic acid, the addition of triisopropylsilane and water in trifluoroacetic acid, or a combination thereof. In an embodiment, the coupling step comprises the addition of the free amino acid building block and coupling solution such as but not limited to solution comprising benzotriazol-1-yloxytripyrrolidinophosphonium hexafluorophosphate (PyBOP), hydroxybenzotriazole (HOBt), diisopropylethylamine (DIEA), diisopropylcarbodiimide (DIC), hexafluorophosphate azabenzotriazole tetramethyl uronium (HATU), hexafluorophosphate benzotriazole tetramethyl uronium (HBTU), HCTU, (7-azabenzotriazol-1-yloxy)tripyrrolidinophosphonium hexafluorophosphate (PyAOP), PyCOP, (1-cyano-2-ethoxy-2-oxoethylidenaminooxy) dimethylamino-morpholino-carbenium hexafluorophosphate (COMU), 1-hydroxy-7-azabenzotriazole (HOAt), or a combination thereof to the amino acid or peptide linked to the resin after the deblocking step.

In an embodiment, the long-chain fatty acid is attached to the first peptide at the N-terminal end of the first peptide. In an embodiment, attaching a long-chain fatty acid to the first peptide comprises a deblocking step and a coupling step. In an embodiment, the deblocking step comprises the addition of deblocking solution such as but not limited to piperidine solution to the amino acid or peptide linked to the resin. In an embodiment, the piperidine solution is supplemented with hydroxybenzotriazole (HOBt). In an embodiment, the deblocking step comprises the addition of palladium catalyst and phenylsilane in dichloromethane, the addition of triisopropylsilane in trifluoroacetic acid, the addition of triisopropylsilane and water in trifluoroacetic acid, or a combination thereof. In an embodiment, the coupling step comprises the addition of the free amino acid building block and coupling solution such as but not limited to solution comprising benzotriazol-1-yloxytripyrrolidinophosphonium hexafluorophosphate (PyBOP), hydroxybenzotriazole (HOBt), diisopropylethylamine (DIEA), diisopropylcarbodiimide (DIC), hexafluorophosphate azabenzotriazole tetramethyl uronium (HATU), hexafluorophosphate benzotriazole tetramethyl uronium (HBTU), HCTU, (7-azabenzotriazol-1-yloxy)tripyrrolidinophosphonium hexafluorophosphate (PyAOP), PyCOP, (1-cyano-2-ethoxy-2-oxoethylidenaminooxy) dimethylamino-morpholino-carbenium hexafluorophosphate (COMU), 1-hydroxy-7-azabenzotriazole (HOAt), or a combination thereof to the amino acid or peptide linked to the resin after the deblocking step.

In an embodiment, the second peptide comprises at least five amino acids such as five or six amino acids. In an embodiment, the second peptide comprises the sixth, seventh, eighth, ninth, tenth, eleventh, twelfth, thirteen amino acid of the modified peptide of the antibiotic of formula (I) of the present invention, or a combination thereof. In an embodiment, the second peptide comprises the sixth, seventh, eighth, ninth, tenth, and eleventh amino acids of the modified peptide of the antibiotic of formula (I) of the present invention.

In an embodiment, attaching the second peptide to the first peptide at an amino acid residue of the first peptide of step d comprises the step of attaching one amino acid building block at a time to an amino acid of the first peptide according to the amino acid sequence of the chemical structure of the antibiotic of formula (I) of the present invention obtained after the chemical structure optimization step. For example, a sixth amino acid building block is attached to the first peptide, and a seventh amino acid building block is attached to the sixth amino acid building block linked to the first peptide. In an embodiment, the amino acid building blocks are attached one by one according to the amino acid sequence of the chemical structure of the antibiotic of formula (I) of the present invention in a C-terminal to N-terminal direction. For example, the sixth amino acid building block attached to the first peptide comprises the eleventh amino acid residue of the modified peptide of the antibiotic of formula (I) of the present invention, and the seventh amino acid building block attached to the sixth amino acid building block comprises the tenth amino acid residue of the modified peptide of the antibiotic of formula (I) of the present invention. In an embodiment, the second peptide is attached to the first peptide at an amino residue located near the N-terminal end of the first peptide. In an embodiment, the second peptide is attached to the first peptide at the second, the third, or the fourth amino acid residue from the N-terminal end of the first peptide.

In an embodiment, the step of attaching one amino acid building block at a time to the amino acid or peptide linked to the resin comprises a deblocking step and a coupling step. In an embodiment, the deblocking step comprises the addition of deblocking solution such as but not limited to piperidine solution to the amino acid or peptide linked to the resin. In an embodiment, the piperidine solution is supplemented with hydroxybenzotriazole (HOBt). In an embodiment, the deblocking step comprises the addition of palladium catalyst and phenylsilane in dichloromethane, the addition of triisopropylsilane in trifluoroacetic acid, the addition of triisopropylsilane and water in trifluoroacetic acid, or a combination thereof. In an embodiment, the coupling step comprises the addition of the free amino acid building block and coupling solution such as but not limited to solution comprising benzotriazol-1-yloxytripyrrolidinophosphonium hexafluorophosphate (PyBOP), hydroxybenzotriazole (HOBt), diisopropylethylamine (DIEA), diisopropylcarbodiimide (DIC), hexafluorophosphate azabenzotriazole tetramethyl uronium (HATU), hexafluorophosphate benzotriazole tetramethyl uronium (HBTU), HCTU, (7-azabenzotriazol-1-yloxy)tripyrrolidinophosphonium hexafluorophosphate (PyAOP), PyCOP, (1-cyano-2-ethoxy-2-oxoethylidenaminooxy) dimethylamino-morpholino-carbenium hexafluorophosphate (COMU), 1-hydroxy-7-azabenzotriazole (HOAt), or a combination thereof to the amino acid or peptide linked to the resin after the deblocking step.

In an embodiment, removing the linear form of the antibiotic of formula (I) of the present invention from the resin comprises the addition of cleaving solution to the resin-linked linear form antibiotic of formula (I) of the present invention. In an embodiment, the cleaving solution comprises trifluoroacetic acid (TFA) in dichloromethane (DCM). In an embodiment, removing the linear form of the antibiotic of formula (I) of the present invention from the resin further comprises the addition of washing solution prior to the addition of the cleaving solution to the resin-linked linear form of the antibiotic of formula (I) of the present invention. In an embodiment, the washing solution comprises DCM.

In an embodiment, circularizing the linear form of the antibiotic of formula (I) of the present invention to form a crude antibiotic of formula (I) of the present invention comprises a coupling step. In an embodiment, the coupling step comprises the incubation of the linear form of the antibiotic of formula (I) of the present invention in a coupling solution such as but not limited to solution comprising hexafluorophosphate azabenzotriazole tetramethyl uronium (HATU), diisopropylcarbodiimide (DIC), benzotriazol-1-yloxytripyrrolidinophosphonium hexafluorophosphate (PyBOP), hydroxybenzotriazole (HOBt), diisopropylethylamine (DIEA), hexafluorophosphate benzotriazole tetramethyl uronium (HBTU), HCTU, (7-azabenzotriazol-1-yloxy)tripyrrolidinophosphonium hexafluorophosphate (PyAOP), PyCOP, (1-cyano-2-ethoxy-2-oxoethylidenaminooxy) dimethylamino-morpholino-carbenium hexafluorophosphate (COMU), 1-hydroxy-7-azabenzotriazole (HOAt), or a combination thereof.

In an embodiment, removing the protecting groups on the amino acid building blocks of the crude antibiotic of formula (I) of the present invention comprises the addition of deprotecting solution to the crude antibiotic of formula (I) of the present invention. In an embodiment, the deprotecting solution comprises TFA, water, triisopropylsilane, or a combination thereof.

In an embodiment, the method of preparation of the antibiotic of formula (I) of the present invention further comprises as step of purifying the antibiotic of formula (I) of the present invention obtained after step g. In an embodiment, the purifying step comprises a HPLC elution.

The present invention further provides a method of inhibiting bacterial growth in a subject or a bacterial culture comprising the step of administration of the antibiotic of formula (I) of the present invention and an atom comprising a boron atom, a calcium atom, or a combination thereof to the subject or the bacterial culture. In an embodiment, the bacteria comprise Gram-positive bacteria, drug-resistant bacteria, or a combination thereof. In an embodiment, the subject is diagnosed with a bacterial infection. In another embodiment, the bacteria are grown in vitro as a bacterial culture. In an embodiment, the method of inhibiting bacterial growth of the present invention comprises the step of administration of the antibiotic of formula (I) of the present invention to the bacterial culture comprising at least a bacteria colony. In an embodiment, the step of administration of the antibiotic of formula (I) of the present invention to the bacterial culture further comprises adding an atom comprising a boron atom, a calcium atom, or a combination thereof. In an embodiment, the boron atom is in the form comprising a boronic acid, boronate, boronic ester, or boronate ester. In an embodiment, boronic acid comprises phenylboronic acid (PBA), 4-hydroxylphenylboronic acid, 3-aminophenylboronic acid, 4-aminophenylboronic acid, 4-cyanophenylboronic acid, 4-methoxyphenylboronic acid, 4-pyridylboronic acid, 3-hydroxylphenylboronic acid, 2-formylphenylboronic acid, 4-(methylthio) phenylboronic acid, 4-methoxycarbonyl phenylboronic acid, 3-pyridylboronic acid, 4-tolylboronic acid, 3,5-dimethylphenylboronic acid, 3,5-(bis(trifluoromethyl)phenyl) boronic acid, 4-fluorophenylboronic acid, or a combination thereof. In an embodiment wherein the bacterial growth environment such as in a subject body or in growth media that already comprises a boron atom, a calcium atom, or a combination thereof, addition of additional a boron atom, a calcium atom, or a combination thereof can be optional.

In an embodiment, the method of inhibiting bacterial growth of the present invention results in complexing between the antibiotic of formula (I), an atom comprising a boron atom, a calcium atom, or a combination thereof, and a bacterial component. In an embodiment, the bacterial component comprises a molecule required for the synthesis of bacterial cell wall. In an embodiment the bacterial component comprises undecaprenyl phosphate, lipid II, or a combination thereof. In an embodiment, the complexing between the CDA derivative of the present invention, an atom comprising a boron atom, a calcium atom, or a combination thereof, and a bacterial component inhibits the synthesis of bacterial cell wall in the bacteria. In an embodiment, the complexing between the CDA derivative of the present invention, an atom comprising a boron atom, a calcium atom, or a combination thereof, and a bacterial component inhibits the bacterial growth.

The present invention further provides a method of treatment of bacterial infection in a subject comprising the step of administration of a therapeutically effective amount of the antibiotic of formula (I) of the present invention to the subject. In an embodiment, the subject is diagnosed with a bacterial infection. In an embodiment, the bacterial infection comprises infection by Gram-positive bacteria, drug-resistant bacteria, or a combination thereof. In an embodiment, the method of treatment of bacterial infection in a subject of the present invention further comprises the step of administration of a therapeutically effective amount of composition comprising boron atom, calcium atom, or a combination thereof. In an embodiment, the composition comprising boron atom, calcium atom, or a combination thereof comprises a boronic acid, boronate, boronic ester, or boronate ester. In an embodiment, the boronic acid comprises phenylboronic acid (PBA), 4-hydroxylphenylboronic acid, 3-aminophenylboronic acid, 4-aminophenylboronic acid, 4-cyanophenylboronic acid, 4-methoxyphenylboronic acid, 4-pyridylboronic acid, 3-hydroxylphenylboronic acid, 2-formylphenylboronic acid, 4-(methylthio) phenylboronic acid, 4-methoxycarbonyl phenylboronic acid, 3-pyridylboronic acid, 4-tolylboronic acid, 3,5-dimethylphenylboronic acid, 3,5-(bis(trifluoromethyl)phenyl) boronic acid, 4-fluorophenylboronic acid, or a combination thereof. In an embodiment, the ratio between the antibiotic of formula (I) and the composition comprising boron atom, calcium atom, or a combination thereof being administered to a subject is about 5:1 to about 1:5 such as about 5:1, about 4:1, about 3:1, about 2:1, about 1:1, about 1:2, about 1:3, about 1:4, about 1:5, or any ratio falling in between these ranges.

In an embodiment, the step of administration of a therapeutically effective amount of the antibiotic of formula (I) of the present invention to the subject is about the same time as the step of administration of a therapeutically effective amount of a composition comprising boron atom, calcium atom, or a combination thereof. In an embodiment, the step of administration of a therapeutically effective amount of the antibiotic of formula (I) of the present invention to the subject is performed about 1 minute, 2 minutes, 5 minutes 10 minutes, 30 minutes or 1 hour before the step of administration of a therapeutically effective amount of a composition comprising boron atom, calcium atom, or a combination thereof. In an embodiment, the step of administration of a therapeutically effective amount of the antibiotic of formula (I) of the present invention to the subject is performed about 1 minute, 2 minutes, 5 minutes 10 minutes, 30 minutes or 1 hour after the step of administration of a therapeutically effective amount of a composition comprising a boron atom, calcium atom, or a combination thereof. In an embodiment, the dosage of the antibiotic of formula (I) of the present invention is about 1 to about 100 mg per kg of the patient's body weight such as about 1 mg per kg, about 2 mg per kg, about 4 mg per kg, about 6 mg per kg, about 8 mg per kg, about 10 mg per kg, about 15 mg per kg, about 20 mg per kg, about 25 mg per kg, about 30 mg per kg, about 35 mg per kg, about 40 mg per kg, about 45 mg per kg, about 50 mg per kg, about 55 mg per kg, about 60 mg per kg, about 65 mg per kg, about 70 mg per kg, about 75 mg per kg, about 80 mg per kg, about 85 mg per kg, about 90 mg per kg, about 95 mg per kg, about 100 mg per kg about 150 mg per kg, or about 200 mg per kg, including any values or value ranges falling within these values. In an embodiment, the antibiotic of formula (I) of the present invention is administered for about 1 to about 8 weeks dependent on the indication such as about 1, about 2, about 3, about 4, about 5, about 6, about 7, or about 8 weeks including any weeks or week ranges falling within these values.

In an embodiment, routes of administering a therapeutically effective amount of the antibiotic of formula (I) of the present invention and/or the composition comprising an atom comprising a boron atom, a calcium atom, or a combination thereof comprise parenteral, e.g., intravenous, intradermal, subcutaneous, oral, transdermal (topical), transmucosal administration or a combination thereof.

It is to be understood that both the foregoing general description and detailed description are exemplary and explanatory only and are not restrictive of the invention, as claimed. In general, the terms used in the disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

### EXAMPLES

### Materials and Methods

### Reagents, Consumables, and Instruments

Solid-phase peptide synthesis (SPPS) was performed using a custom-made fritted glass reaction vessel (RV). Amino acid building blocks and coupling reagents were purchased from P3Biosystem, BLDpharm, and AgeneMax; phenylboronic acid (PBA) was purchased from BLDpharm; geranyl monophosphate (C10P) lithium salt was purchased form Sigma-Aldrich; other chemicals were purchased from Merck, ThermoFisher Scientific, J. T. Baker, and Uni-Onward Corp. All chemicals are of ACS grade (or higher) and used as is. Growth media and nutrient supplements were prepared from premixed powders purchased from the following vendors: lysogeny broth (LB, BioShop Canada Inc.), tryptic soy broth (TSB, HiMedia Laboratories LLC), brain heart infusion (BHI, Neogen Corp.), beef extract (Sisco Research Laboratories), peptone (BioShop Canada Inc.). Dulbecco's Modified Eagle Medium (DMEM), Phosphate buffered saline (PBS), antibiotic-antimycotic solution (100×), 0.25% Trypsin buffer, and Fetal Bovine Serum (FBS) were purchased from Gibco. 3-(4,5-Dimethylthiazol-2-yl)-2,5-Diphenyltetrazolium Bromide (MTT) was purchased from Invitrogen. Peptides were purified using a C18 semi-preparative column (HYPER GLD AQ PREP, 5 μm, 250×10 mm, ThermoFisher Scientific) on a Waters HPLC Instrument (996 UV detector, 600 pump and controller) using a two-solvent gradient system, wherein solvent A is water with 0.1% (v/v) formic acid and solvent B is acetonitrile (ACN) with 0.1% (v/v) formic acid. TLC mobility shift assays were performed on TLC Silica Gel 60 F254 (Merck). Mass spectra of synthetic peptides were acquired by using ESI-TOF (microTOF-QII, Bruker), and the formation of various peptide complexes was monitored by NESI-HRMS on a high-field mass spectrometer (Orbitrap Elite Hybrid, ThermoFisher Scientific). All NMR spectra were acquired on a Bruker AVIII 400 (400 MHZ) instrument. MTT assay results were read by microplate reader (EPOCH 2, BioTek).

### Synthesis of Fmoc-D-allo-Thr-OH

D-allo-Thr-OH (1.53 g) and Na₂CO₃ (2.67 g) were dissolved in water (20 mL). Fmoc-OSu (5.42 g) was dissolved in ACN (20 mL), added dropwise to the above solution, and stirred overnight. White precipitates formed during the course of this reaction. ACN was removed under reduced pressure, and the solution was acidified to pH 4 by 1 N HCl and extracted by ethyl acetate (EA, 100 mL, 3×). The combined EA layers were washed with brine (3×) and dried under vacuo. The resulting white powder was then recrystallized in an EA/hexane mixture and used in SPPS without further purification (3.52 g, 80.6% yield). TLC (EA/Hex=5:1) R_{f}=0.1. ¹H NMR (400 MHZ, MeOD) δ 7.78 (d, J=7.5 Hz, 2H), 7.67 (dd, J=7.5, 3.3 Hz, 2H), 7.39 (t, J=7.5 Hz, 2H), 7.31 (dt, J=7.5, 1.1 Hz, 2H), 4.36 (m, 2H), 4.25 (t, J=7.0 Hz, 1H), 4.21 (d, J=6.9 Hz, 1H), 4.12 (quintuplet, J=5.9 Hz, 1H), 1.25 (d, J=6.4 Hz, 3H); ¹³C NMR (400 MHZ, MeOD) & 172.35, 157.19, 143.78, 143.70, 141.06, 127.29, 126.67, 124.74, 119.42, 67.32, 66.64, 59.80, 17.96. ESI-HRMS calcd for C₁₉H₁₉NNaO₅⁺, 364.1155; found [M+Na]⁺: 364.1146.

### Solid-Phase Peptide Synthesis—General Methods

SPPS began with the loading of Asp5 onto 2-chlorotrityl resins (CTC, 1.50 mmol/g) that had been pre-swollen in dichloromethane (DCM) for 30 minutes. A solution containing Fmoc-Asp(tBu)-OH (2 equiv.) in DCM and DIEA (4 equiv.) was added to the resins, and the mixture was placed on an orbital shaker set at 180 rpm for 16 hours. Following DCM washes (5×), a DCM solution of diisopropylethylamine (DIEA, 5% (v/v)) and methanol (MeOH, 10% (v/v)) was added to the resins and shaken for 1 h to cap the remaining unreacted sites. The resins were then washed with DCM (3×) and DMF (3×). Removal of the Fmoc protecting group was carried out using 20% (v/v) piperidine in DMF to determine the loading yield based on the reported extinction coefficient of the Fmoc-piperidine adduct (7,800 M⁻¹ cm⁻¹). The observed loading yield was defined as one equivalent and typically in the 60 to 80% range (0.90 to 1.20 mmol/g).

SPPS was performed using amino acid building blocks compatible with standard Fmoc chemistry. Each cycle includes deblocking and coupling steps. For the deblocking step, the resins underwent two rounds of 20% piperidine in DMF (v/v) treatment for 5 and 15 minutes. For the coupling step, the Fmoc building blocks (3 equiv.), benzotriazol-1-yloxytripyrrolidinophosphonium hexafluorophosphate (PyBOP, 3 equiv.), hydroxybenzotriazole (HOBt, 3 equiv.), and DIEA (8 equiv.) were pre-mixed in DMF (approximately 2 mL), added to the resins, and allowed to react at room temperature for 1 h. Following each deblocking and coupling steps, the resins were washed with DMF (5×). The N-terminal palmitoyl chain was coupled using the same procedures as described above. Note that the D-allo-Thr-OH building block was used with no side-chain protection, and the side-chain amine (—NH₂) of the Dap2 building block was protected by allyloxycarbonyl (Alloc) group.

### Preparation of Synthetic LspC Analogs

Starting from CTC resins loaded with Asp5, amino acids building blocks were coupling in the following order: Gly4, pip3, Dap2, Ser1/Hse1, and the N-terminal palmitoyl modification. Palladium catalyst (Pd(PPh₃)₄, 20 mol %) and phenylsilane (20 equiv.) in DCM was added to remove the Alloc protecting group on Dap2; this step was performed twice (30 min per round) under argon atmosphere. SPPS then continued by coupling amino acid building blocks in the following order: Pro11, Ile 10, D-allo-Thr9, Gly8, Ser7/Hse7, and Gly6. Note that the piperidine solution was supplemented with 5% (w/w) HOBt to minimize aspartimide formation during the deblocking step after the coupling of an Asp residue. Once the entire peptide backbone is completed, the resins were washed with DCM (5×) and cleaved from the CTC resins into a 250 mL round bottom flask by treating the resins with 1% (v/v) trifluoroacetic acid (TFA) in DCM (2 mL, 5×). The solution was then diluted to 125 mL, followed by the addition of HATU or N,N-diisopropylcarbodiimide (DIC, 1 mmol), HOBt (1 mmol) and DIEA (500 μL). The reaction was stirred for two days and the solution was concentrated under reduced pressure. A TFA solution (10 mL) containing 2.5% (v/v) each of water and triisopropylsilane was used for cleavage and global deprotection (1 h). After removal of TFA, the crude was redissolved in 1:1 H₂O/ACN (3 mL) and purified by HPLC. All of our synthetic LspC analogs were eluted in the 65 to 70% B range from a C18 column, wherein solvent A and B are formic acid supplemented (0.1% (v/v)) water and ACN, respectively. Sample was collected, lyophilized, and the resulting powder was stored at −20° C. (2 to 8% yield).

### Determination of the Minimum Inhibitory Concentration

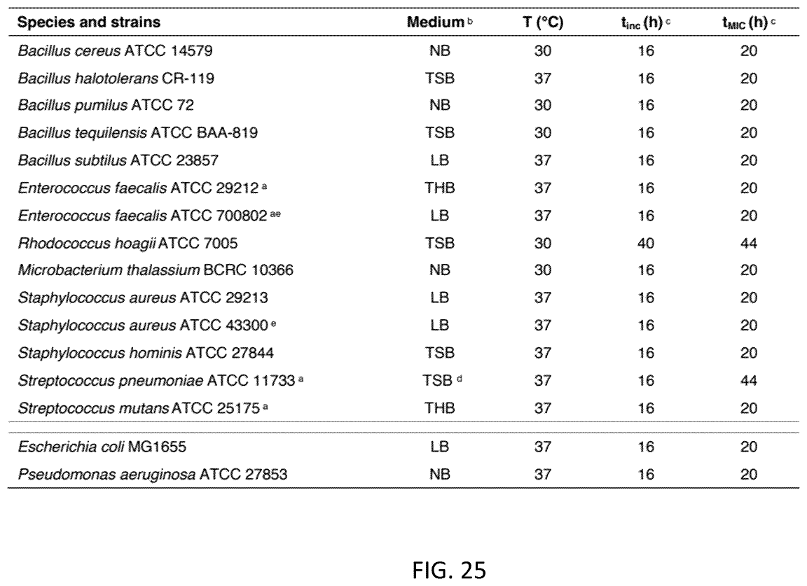

A single bacterial colony on an agar plate was inoculated into the appropriate growth medium, grown to stationary phase, diluted 5,000-fold, and used as the inoculum to setup the MIC assay. The peptide of interest was added to the growth medium to generate a working solution (256 μg/mL), which was used to generate a two-fold dilution series across a 96-well microtiter plate (50 μL per well). Well 1 to 10 contained the dilution series and the last two wells were reserved for positive (no peptide) and negative (no bacteria) controls. Bacterial inoculum was then added to each well (50 μL), so that the final peptide concentrations ranged from 128 to 0.25 μg/mL. The microtiter plate was incubated statically prior to readout; incubation conditions are specified in FIG. 25. To determine the MIC of a peptide in the presence of supplement(s), e.g., calcium (CaCl₂)), phenylboronic acid (PBA), and/or geranyl phosphate (C10P), the supplement(s) was premixed with the growth medium at the appropriate concentration and used as the diluent to prepare the peptide dilution series.

### Thin Layer Chromatography (TLC)

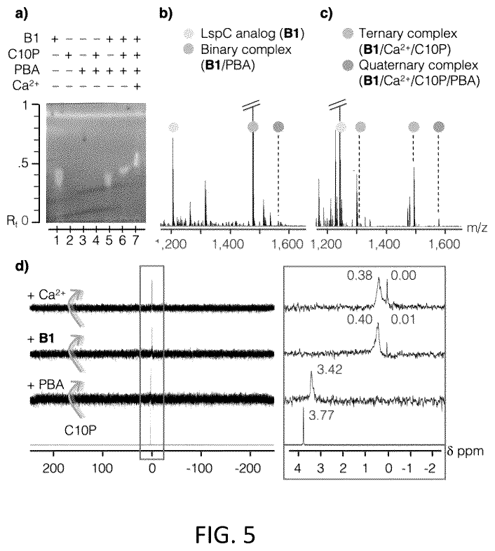

To perform the TLC mobility shift assay shown in FIG. 5A, the following stock solutions were prepared in water: B1 (2.8 mg/mL), PBA (0.4 mg/mL), C10P (0.8 μM) and CaCl₂·2H₂O (40 mM). They were mixed and spotted on a TLC plate as follows. Lane 1: B1; Lane 2: C10P; Lane 3: PBA; Lane 4: C10P/calcium 1:4 (v/v) mixture; Lane 5: B1/PBA/H₂O 1:1:4 (v/v) mixture; Lane 6: B1/PBA/C10P/H₂O 4:4:1:4 (v/v) mixture; Lane 7: B1/PBA/C10P/calcium 4:4:1:4 (v/v) mixture. Note that B1 was the limiting reagent in all of the mixtures. The TLC assay was carried out using a 2:1 (v/v) mixture of DCM/MeOH as the mobile phase and stained by KMnO₄ to enhance contrast.

### Mass Spectrometry (MS)

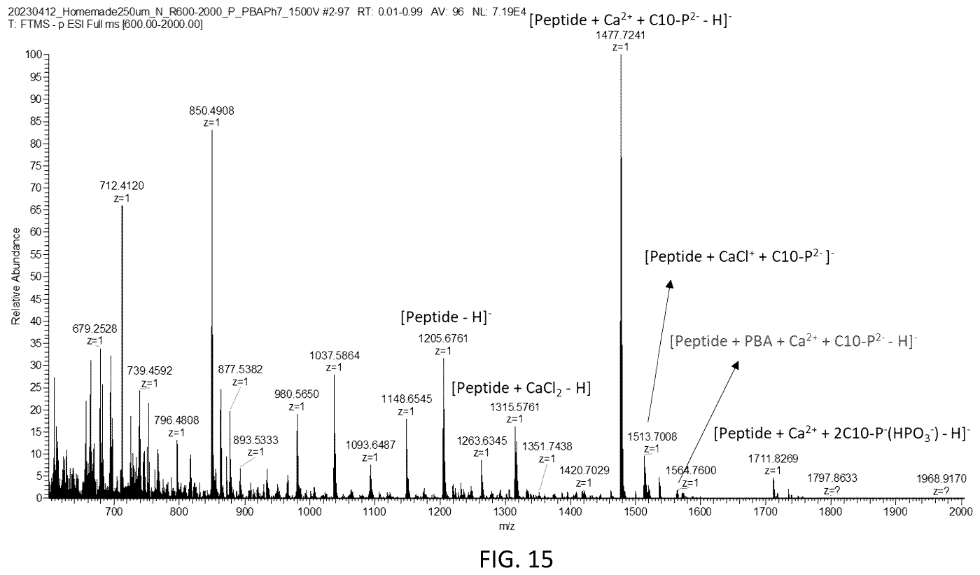

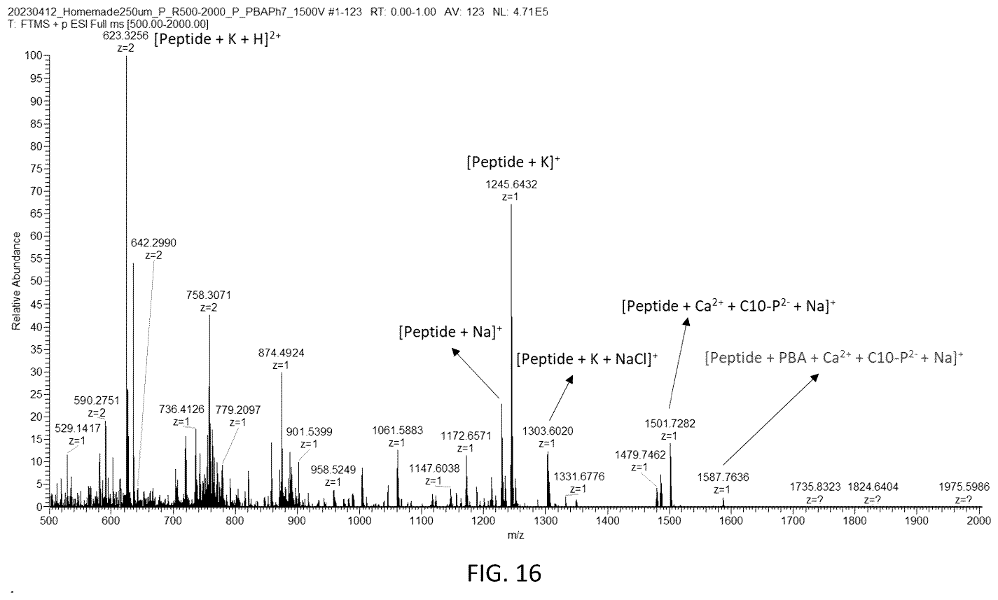

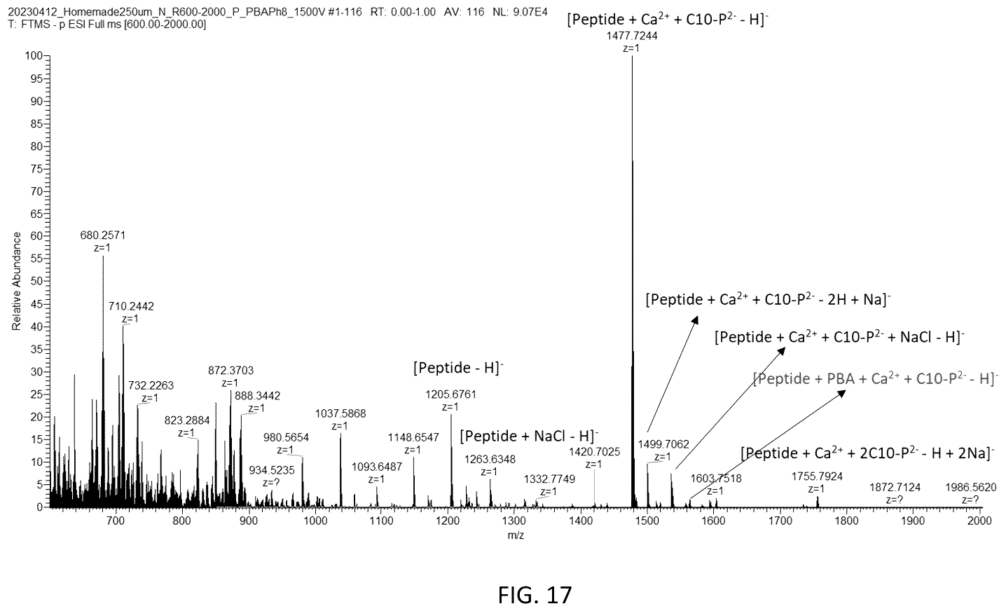

Native electrospray ionization high-resolution mass spectrometry (NESI-HRMS) was used to monitor B1 complex formation as shown in FIGS. 5B and 5C. This is a soft ionization method often used to monitor noncovalent interactions, such as protein-protein and protein-ligand complexes. Since the B1 complex is likely held together by a boronate ester, which forms spontaneously and reversibly in aqueous environment, we decided to use NESI-HRMS to detect the potentially delicate complex. A 1:50:2:2 (molar ratio) mixture of B1, PBA, C10P, and CaCl₂) was prepared in water, and then lyophilized. A solution containing 10 mM NH₄Cl and 100 mM PBA was prepared and adjusted to pH 7.0 and 8.0. Samples were prepared immediately before MS analysis by dissolving lyophilized B1 powder (2.0 mg) in either the pH 7 or pH 8 buffer (0.8 mL). NESI-HRMS was performed by direct infusion with an Orbitrap Elite instrument coupled with a handmade glass capillary (I.D. ˜ 250 μm, emitter ˜30 μm) as the ESI source. Spectra were obtained in both positive and negative ionization modes, wherein the flow rate, capillary spray voltage, and probe temperature, were set at 6 μL/min, 1.5 kV, and 280° C., respectively, with a 120,000 resolution and 600 to 2,000 m/z scan range. Four datasets were acquired as we tested combinations of both buffer (pH 7 and 8) and ionization mode (positive and negative); the spectra are shown in FIGS. 14-17 and peak assignments are shown in FIGS. 24-25. Note that salt suppression, ionization efficiency, and complex stability may affect signal intensity to different extents, so that observed peak height is generally not representative of the relative abundance of complexes in the solution

### Nuclear Magnetic Resonance (NMR)

NMR experiments were conducted on a Bruker AVIII 400 (400 MHZ) spectrometer. Chemical shifts (δ) are relative to tetramethylsilane, boric acid, and phosphoric acid for ¹H, ¹¹B, and ³¹P spectra, respectively. The ¹¹B spectrum of PBA (20 μL, 10.0 mg/mL) in 10% D₂O (550 μL) was first obtained. Next, C10P (17 μL, 16 mg/mL) was added directly into the NMR tube, and both the ¹¹B and ³¹P spectra were obtained. B1 (1.2 mg, powder) was then added, followed spectra acquisition. Finally, CaCl₂) was added to the solution in the NMR tube, so that the final calcium concentration is ˜5 mM. Note that the ³¹P spectrum of C10P alone was collected separately.

### Molecular Dynamics Simulation

The initial structure of the B1/PBA/C10P/Ca²⁺ complex was prepared using PyMol based on the co-crystal structure of LspC in complex with calcium and C10P (PDB ID: 5O0Z). In 5O0Z, only a small portion of the fatty acyl chain could be resolved. The structure was then imported to Schrodinger Maestro,¹ where hydrogen atoms were added, and the double bond in the fatty acyl chain was changed to a single bond as in B1. The ffld_server feature was used to prepare the topology with the OPLS-2005 forcefield.² A Python program, ffconv.py,³ was used to convert the generated topology from the Schrodinger Maestro format to the GROMACS format. GROMACS 2018.8 was used for all the subsequent procedures and molecular dynamics (MD) simulations.⁴ The initial B1/PBA/C10P/Ca²⁺ complex structure was first energy minimized in vacuum using the steepest descent algorithm. The energy-minimized complex was then solvated with pre-equilibrated TIP4P water box.⁵ The distance between the B1/PBA/C10P/Ca²⁺ complex to the box walls was at least 1.0 nm. One Na⁺ was added to the solvated complex to neutralize the whole system. The resulting system was energy minimized using the steepest descent algorithm. Five independent sets of MD simulation with different initial velocities were performed starting from this structure.

For each of the five sets of MD simulation, a four-step equilibration was performed. The first two equilibration steps were a 50 ps isothermal-isochoric (NVT) simulation and a 50 ps isothermal-isobaric (NPT) simulation. In these two equilibration steps, a harmonic position restraint with a force constant of 1,000 kJ/(mol·nm²) was applied to each heavy atom of the complex. The last two equilibration steps were a 100 ps NVT simulation and a 100 ps NPT simulation without the position restraints to equilibrate the whole system. The v-rescale thermostat with a time coupling constant of 0.1 ps was used to keep all NVT and NPT simulations at 300 K. The Parrinello-Rahman barostat with a time coupling constant of 2.0 ps and isothermal compressibility of 4.5×10⁻⁵ bar⁻¹ was used to keep NPT simulations at 1 bar. Throughout each four-step equilibration process, the LINCS constraint algorithm was applied to all bonds. The periodic boundary conditions were used in all directions of the simulation box. Both Lennard-Jones and electrostatic interactions were truncated at 1.0 nm. The particle mesh Ewald was applied for electrostatic interactions beyond the cutoff distance with a Fourier spacing of 0.12 nm and an interpolation order of 4. The long-range dispersion correction for energy and pressure was applied for Lennard-Jones interactions beyond the cutoff. The leapfrog algorithm with a time step of 2 fs was used to integrate the dynamic equations of the system. After the equilibration, 100 ns of NPT simulation was performed as the production run. All MD parameters of these production runs were kept unchanged as described in the NPT equilibration, except for one parameter: the LINCS constraint algorithm was now applied to only bonds involving hydrogen atoms.

### Cytotoxicity Assays

HEK293T cells were cultured in Dulbecco's Modified Eagle's Medium supplemented with 10% fetal bovine serum and 1% Antibiotic antimycotic at 37° C. under humidified atmosphere and 5% CO₂. Cytotoxicity assays began by seeding 2×10⁴ HEK293T cells per well in a 96-well plate. Cells were given 20 to 24 h to adhere, washed with fresh medium, and then a 2-fold dilution series of the compounds of interest were added. The highest concentration tested for S1, B1, and PBA were 128, 128, and 1,280 μg/mL, respectively; for the B1-plus-PBA series, the PBA concentration was held constant at 5 μg/mL while the B1 concentration varied. After incubation for 24 h, the medium was discarded, the cells rinsed with PBS buffer, and 100 μL of 0.5 mg/mL MTT in PBA was added to each well. The plate was incubated for 2 h before discarding the MTT solution. The cells were wash with PBS, and then the resulting purple crystals were dissolved by DMSO (100 μL per well). Absorbance at 570 nm for each well was recorded, and cell viability (%) was calculated as the ratio of the absorbance the compound-treated well vs. the no-compound well. This assay was performed in triplicate.

### Results

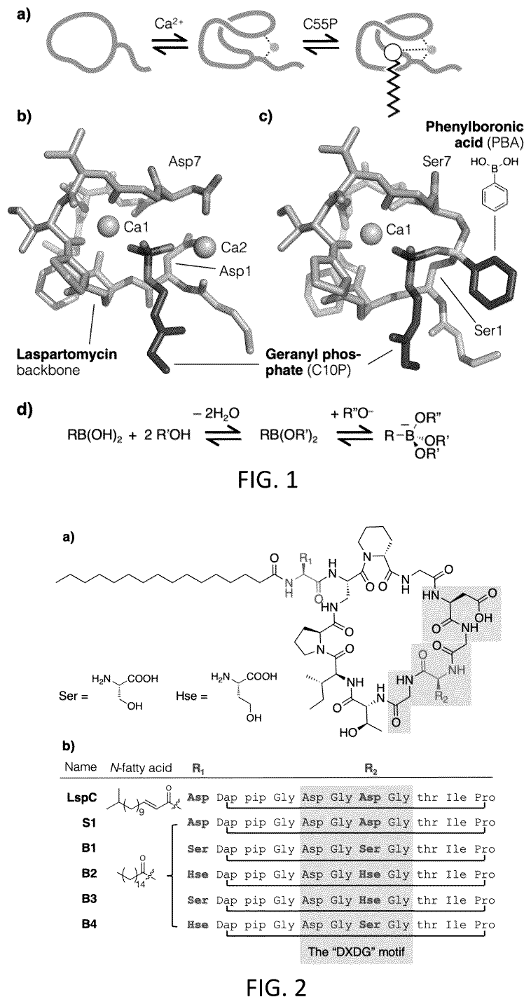

The crystal structure of LspC shows that it is in complex with two calcium and one geranyl phosphate (C10P), a surrogate of the actual substrate C55P (FIG. 1B).^{[10a, 11b]} Together, the amide carbonyl of residue 2, 6, 8, and 10 and the side-chain carboxylate of Asp5 form a binding pocket for the “internal” calcium (Ca1). The “external” calcium (Ca2) is surrounded by the side-chain carboxylate of Asp1 and Asp7, as well as the carbonyl of the N-fatty acyl chain. The Ca2 not only stabilizes LspC in a bicyclic conformation, but also engages the negatively charged phosphate head group of C55P. Sequestration of C55P by LspC disrupts bacterial cell wall biosynthesis to suppress bacterial growth, and it appears that the conformational change critical to LspC activation is mediated by Ca2. Based on this notion, we hypothesized that Ca2 could be replaced by a molecular entity, which does not necessarily need to be a metal cation, that functions as an anchor to bring the C55P phosphate head group and the side-chains of residue 1 and 7 into close proximity to secure the peptide in the active conformation (FIG. 1C).

### Design Rationale for Laspartomycin Derivatives

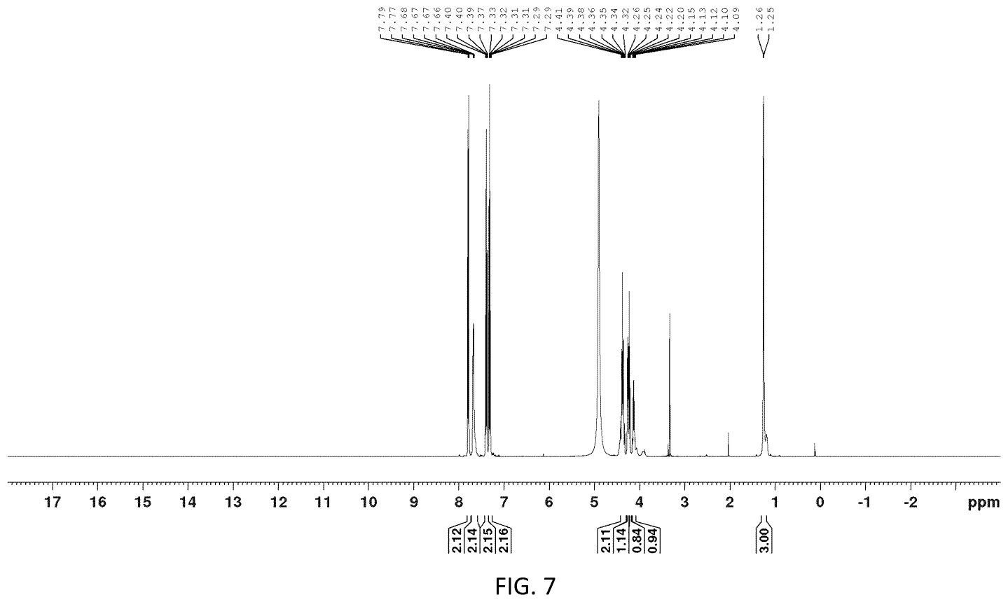

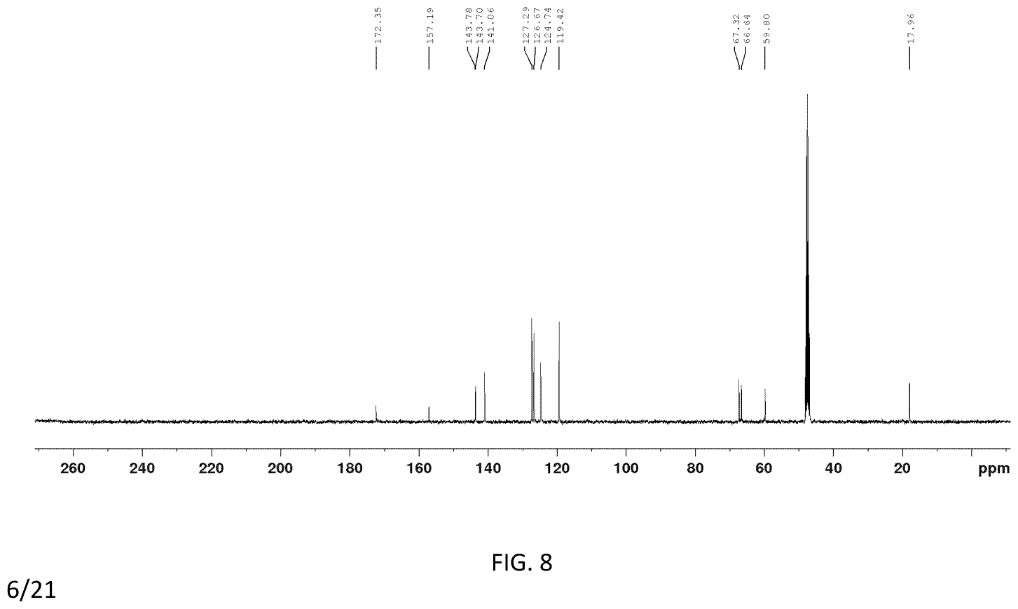

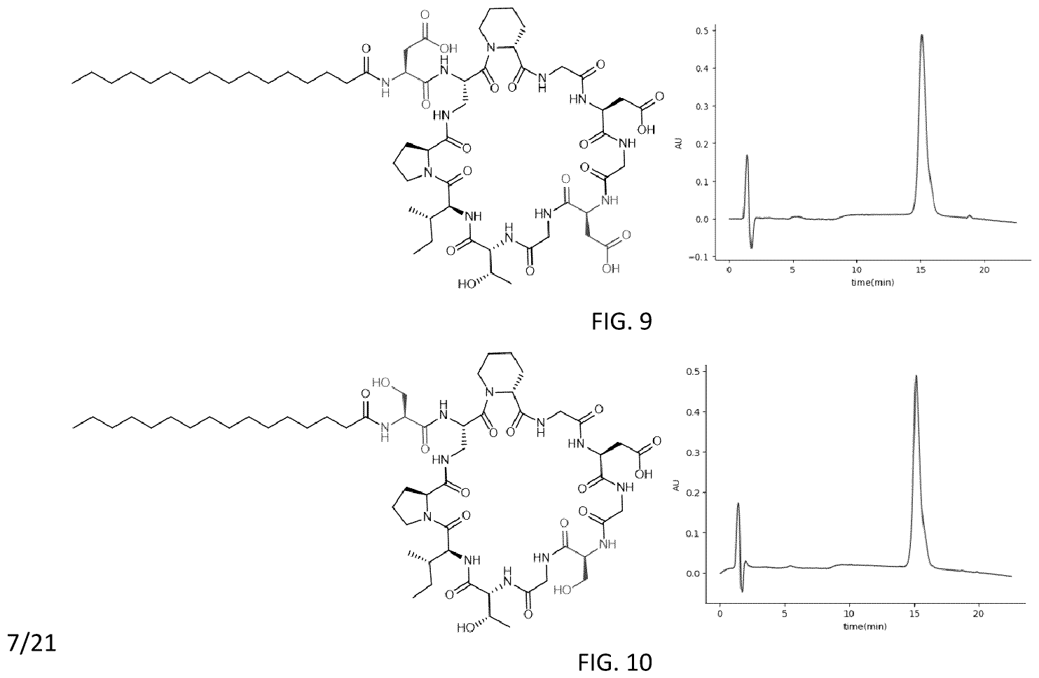

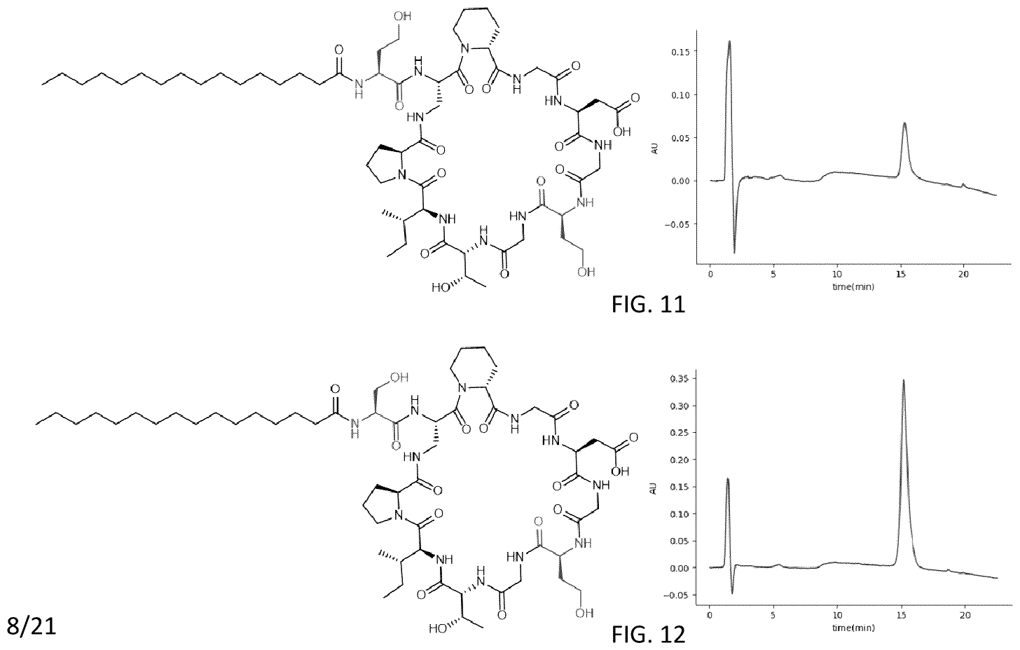

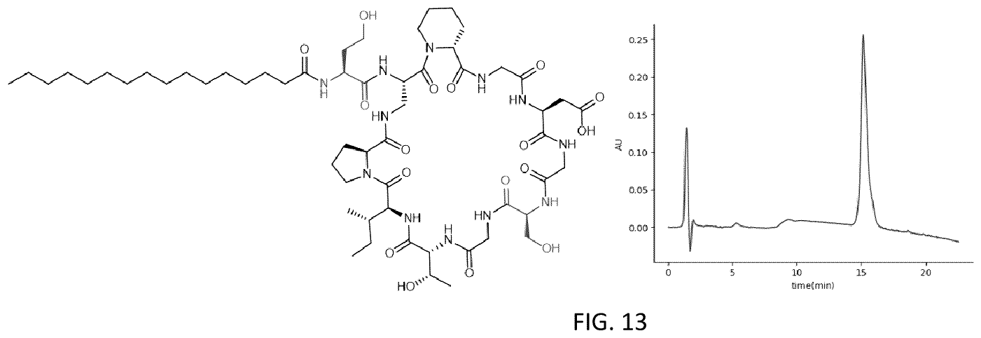

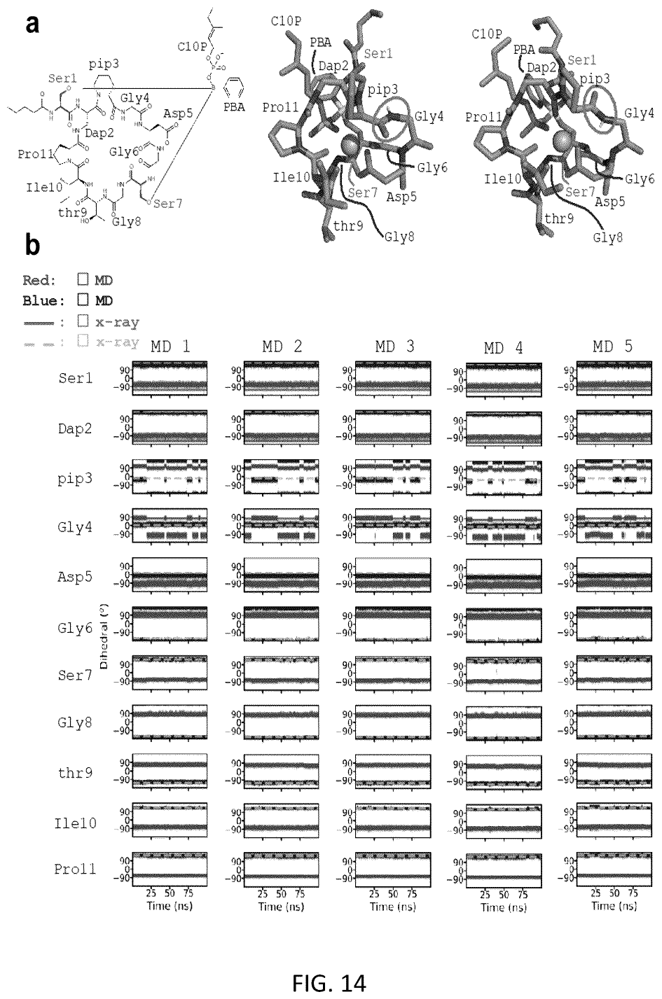

In light of the considerations outlined above, boronic acids come to mind as a class of molecular entity that may serve as the anchor. As they are generally not cytotoxic to mammalian cells, boronic acids have long been a useful element in medicinal chemists' toolkit.^{[12]} A boronic acid can condense with two alcohol molecules to form a boronic ester spontaneously and reversibly. In addition, the boron atom in a boronic acid has an empty p orbital to accept lone pair electrons on oxyanions to form a boronate ester (FIG. 1D). Furthermore, these reactions occur readily under physiological conditions, i.e., in an aqueous solution at near neutral pH and ambient temperature. In fact, several drugs currently in clinical use contain boron, e.g., the anticancer drugs bortezomib and ixazomib, the metallo-β-lactamase inhibitor vaborbactam, the fungal leucyl-tRNA synthetase inhibitor tavaborole, etc. Our synthetic LspC analog designs thus entail replacing the side-chain carboxylates of residue 1 and 7 in LspC with hydroxyl groups (FIG. 2A). For these compounds, we envisioned that the presence of a boronic acid shall induce and secure a conformational change similar to what calcium does for LspC, thereby unleashing the full antimicrobial capacity of our peptide. The spatial location of the boronic acid anchor relative to the peptide backbone is expected to be crucial for inducing the appropriate conformational change, and therefore we decided to synthesize a series of four peptides (B1 to B4) that encompasses at residue 1 and 7 all combinations of serine (Ser) and homoserine (Hse) (FIG. 2B and FIG. 7-13). Ser and Hse both harbor a side-chain hydroxyl group that can condense with a boronic acid to form a boronic ester. They differ by only a single methylene unit (—CH₂—) and can fine-tune the spatial positioning of the boronic ester anchor by just as much. Peptide S1 was also synthesized as a positive control. N-terminal modifications do not change the activity of a CDA as long as the substituent remains sufficiently hydrophobic.^{[10e, 13]} It is therefore well justified to use S1 as a surrogate of LspC since these two compounds are identical except for a minor difference in their N-terminal fatty acyl chain.

To test whether our designs may secure the desired conformation, we built a model of the quaternary complex B1/PBA/C10P/Ca²⁺ based on the structure of the co-crystallized LspC/C10P/Ca²⁺ complex (PDB ID: 5O0Z). Using this structure as a starting point, we performed five runs of molecular dynamics (MD) simulation with different initial velocities using the OPLS-2005/TIP4P force field (see Supporting Information for more details).^{[14]} All five simulations showed that, except for an amide flip between pip3 and Gly4, which was not directly involved in C10P and calcium binding, the backbone of the cyclic peptide remained stable throughout the 100 ns MD simulations (FIG. 1C and FIG. 14).

### Initial Assessments of Peptides B1 to B4

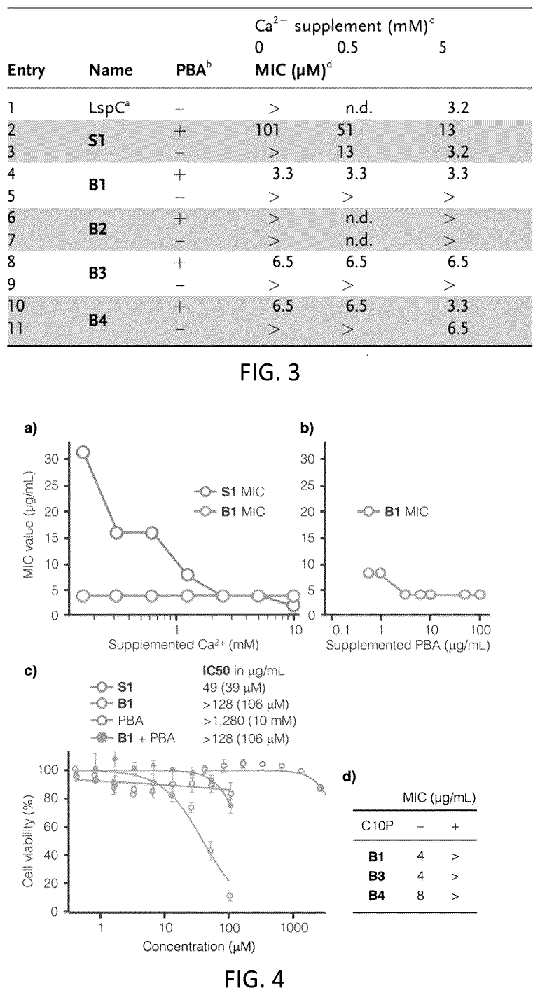

The antimicrobial activities of our synthetic LspC analogs were first assessed against Bacillus subtilis (FIG. 3). We determined their minimum inhibitory concentration (MIC) in media supplemented with calcium at a concentration LspC reportedly showed high antimicrobial capacity (5 mM).^{[9]} As expected, the control peptide S1 was as potent as LspC at this condition (MIC 4 μg/mL (3.3 μM), entry 1 and 3). Nevertheless, most of its activity was lost at [Ca²⁺]=0.5 mM (MIC 16 μg/mL (13 μM)) and became inactive without calcium supplement (MIC>128 μg/mL (104 μM)). Phenylboronic acid (PBA) was chosen as the boron species for its high solubility in water and low cytotoxicity. Interestingly, the potency of S1 dramatically decreased in the presence of PBA (100 μg/mL (0.82 mM), entry 2). This result suggests that PBA interacts with S1 in a way that interferes with C10P sequestration and highlights the challenge to engineer a peptide with PBA inducible bioactivity. Peptide B2 remained inactive in all assays (entry 6 and 7). In contrast, peptide B1, B3, and B4 were about as potent as S1 (MIC 4 to 8 μg/mL (3.3 to 6.6 μM)) in suppressing bacterial growth in the presence of PBA (entry 4, 8, and 10). The importance of the boron anchor (PBA) is evident when comparing entries 4, 8, and 10 to entries 5, 9, and 11, respectively. First, none of them were active in the absence of PBA when the growth media contained no calcium supplements. Second, an order-of-magnitude drop in calcium concentration from 5 to 0.5 mM resulted in a significant loss of activity for S1 (MIC from 4 to 16 μg/mL (3.3 to 13 μM), entry 3), whereas the MICs of B1, B3, and B4 were unaffected as long as PBA was present.

### Quantitation of the Dependence on Calcium and PBA

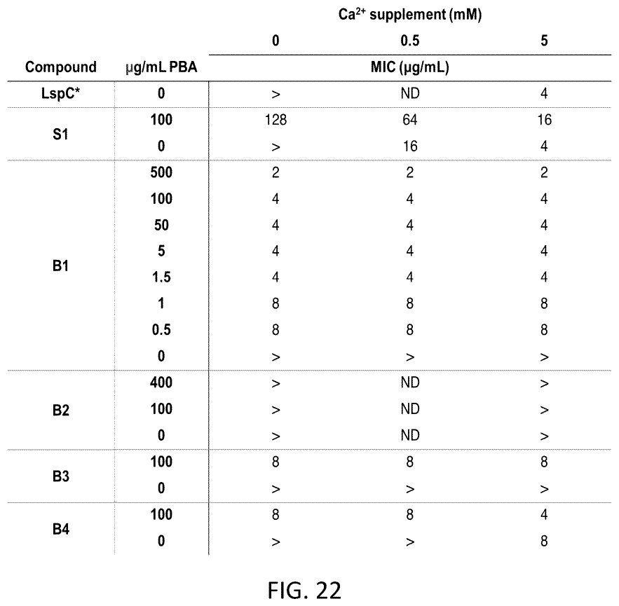

We next assessed in greater detail the extent to which the antimicrobial activity of S1 and B1 were influenced by changes in calcium concentration (FIG. 4A and FIG. 22). LspC is a potent antibiotic with MICs in the single digit μg/mL range at high calcium (≥1.25 mM). Consistent with literature reports,^{[9a]} S1 became less and less effective as the calcium concentration decreased, manifested by a steady increase in MIC. On the other hand, the antimicrobial activity of B1 in the presence of fixed PBA (100 μg/mL (0.82 mM)) showed no change in response to varying calcium concentration. In fact, B1 was equally effective when no calcium was added. Since calcium is essential for bacterial growth and is present in all microbial culture media, the MIC of B1 could not be determined under conditions completely free of calcium. Nevertheless, we showed using other analytical methods (TLC, MS, NMR) that C10P sequestration, to which bacterial growth inhibition is attributed, is independent of calcium (see below).

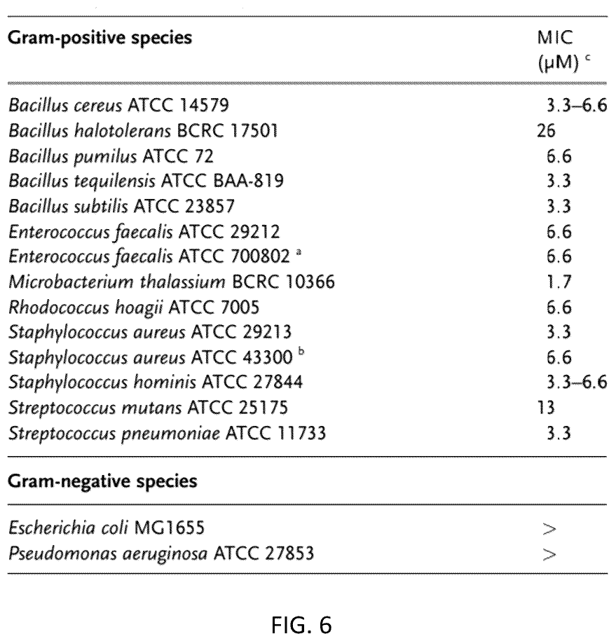

We then investigated the boron dependence of B1 by determining the MIC of B1 across more than three orders of magnitude of PBA concentrations (0.5 μg/mL to 100 μg/mL, FIG. 4B). The data showed that about one equivalent of PBA (0.5 μg/mL (4.1 uM)) was enough to activate B1 nearly to its full capacity (MIC 8 μg/mL (6.6 uM)). This means that the formation of the boronate ester in the B1/PBA complex is highly specific and extremely stable under physiological conditions—neutral pH, ambient temperature, and in an aqueous environment. Furthermore, PBA and our synthetic LspC analogs show very low cytotoxicity (FIG. 4C). The IC50 of S1 against HEK293T cells is 49 μg/mL (39 μM), and the IC50s of B1 and PBA, either alone or in combination, are all greater than 128 μg/mL (the highest concentration tested). The cytotoxic concentrations of B1 and PBA are nearly two orders of magnitude higher than what is needed to suppress the growth of bacterial pathogens (MIC 2 to 8 μg/mL (1.6 to 6.6 μM), FIG. 6), potentially providing a wide therapeutic window.

### Mechanism of Action Studies

Early mechanistic studies of LspC showed that it sequesters the bacterial cell wall biosynthesis intermediate C55.^{[9a]} Because C55P is difficult to handle due to its amphiphilic nature, scientists often use organophosphates with a shorter hydrocarbon chain, e.g., geranyl phosphate (C10P), as a surrogate in model studies.^{[10a, 11b]} As described above, the MOA of LspC inspired the design of our synthetic analogs and to test whether they function in the same way as that of LspC, the MICs of peptide B1, B3, and B4 were determined in growth media supplemented with C10P. None of them were able to inhibit bacterial growth in the presence of an excess amount of C10P, suggesting that C10P competed for binding to our peptides and prevented them from sequestering the natural substrate C55P to disrupt cell wall biosynthesis (FIG. 4B). Bacterial growth was therefore unaffected.

We then sought direct evidence of C10P interaction with our LspC inspired peptide derivatives by using a thin layer chromatography (TLC) assay (FIG. 5A). In this assay, distinct molecular species often differ in mobility, and the formation of a new complex would manifest as a shift in mobility compared to its individual molecular constituent. Peptide B1 was tested in combination with various additives. First, we mixed B1 with PBA and saw no apparent change in mobility as compared to B1 alone (R_{f}˜0.4), though the possibility that B1 and its PBA complex coincidentally display the same mobility could not be ruled out. The addition of C10P, and then calcium, to the B1/PBA mixture both resulted in clear mobility shifts (DR_{f}+0.1 each), which strongly supports the formation of a B1/PBA/C10P ternary complex and a B1/PBA/C10P/Ca²⁺ quaternary complex.

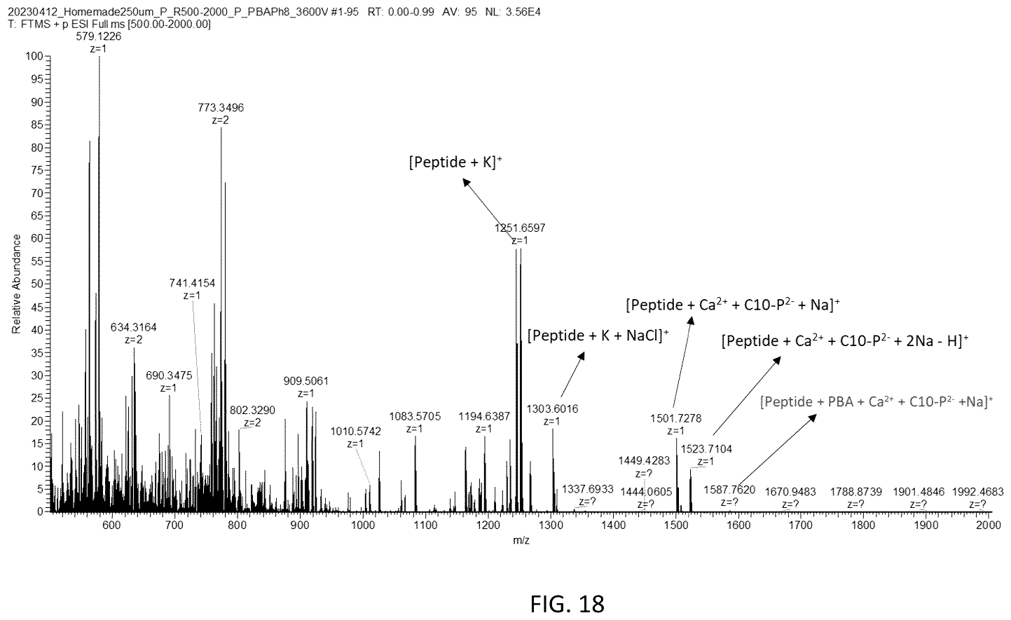

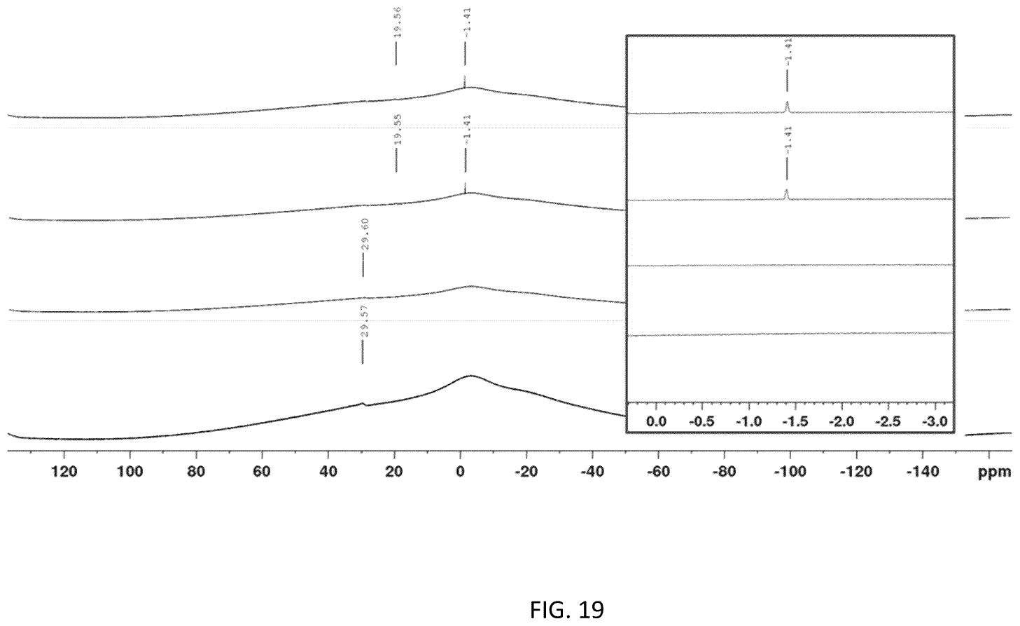

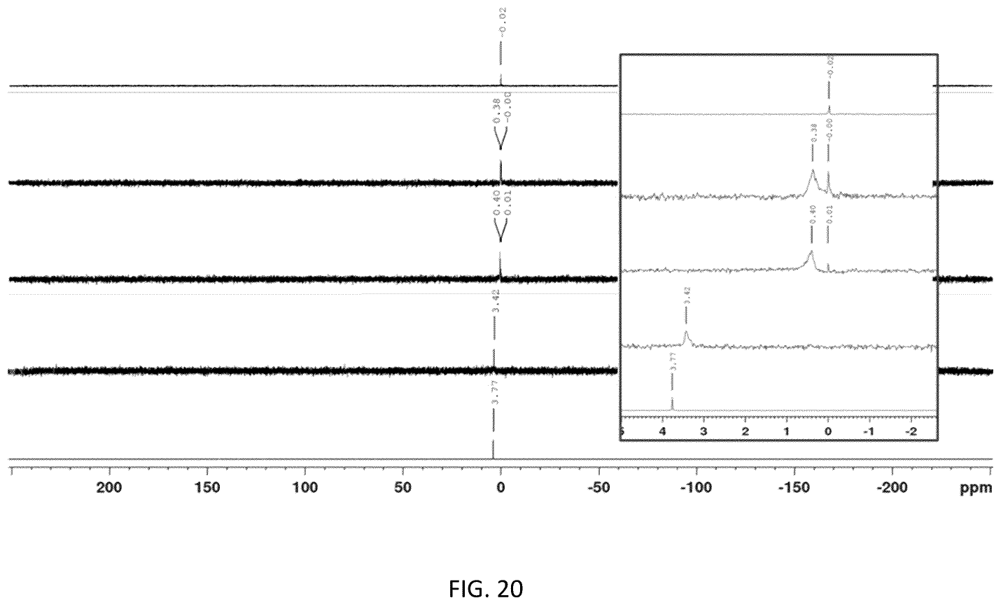

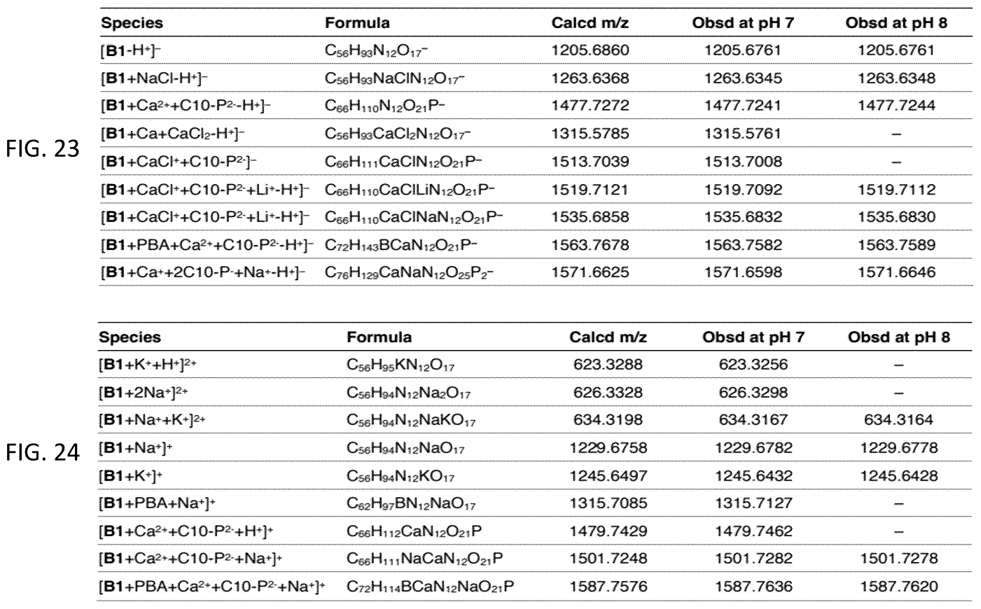

Mass spectrometry (MS) and nuclear magnetic resonance (NMR) offered additional evidence of complex formation at the molecular level. High-resolution native electrospray ionization mass spectrometry (HR-NESI-MS) is commonly used to study noncovalent interactions.^{[15]} Using HR-NESI-MS, we observed the expected m/z for binary, ternary, and quaternary complexes at both the positive and negative modes (FIGS. 5B and 5C, FIGS. 15-18 and FIGS. 23-24). We then performed phosphorous (³¹P) and boron (¹¹B) NMR to monitor the complex formation process. The chemical shift of a nucleus in an NMR spectrum reflects its chemical environment and can be used to detect subtle changes in its immediate surroundings. Our ³¹P NMR experiments began with evaluating C10P alone in 10% D₂O buffer, which showed a single peak at δ 3.7 (FIG. 5D and FIGS. 19-20). There was a small change in chemical shift upon the addition of PBA (δ 3.4), which may be attributed to a slight change in pH upon the addition of a weak acid (PBA). When B1 was added to the C10P/PBA mixture, no phosphorous signal could be detected at δ 3 or greater, and two new peaks at δ 0.4 and 0.0 appeared. The stochiometric ratio of B1/PBA/C10P at this stage was 1:2:1. These observations suggest that B1 had sequestered all C10P in the solution and formed two types of slightly different ternary complexes, likely the boronic and boronate esters. Finally, the phosphorous signals remained practically unchanged upon adding an excess amount of Ca²⁺ (5 mM), suggesting that the presence of Ca²⁺ had little, if any, effect on the B1/PBA/C10P ternary complex. Presumably, the bound calcium occupied the Ca1 position and is too distant from the phosphorous atom to influence its chemical shift. Interestingly, all complexes eventually equilibrated to a single peak at δ 0.00 (FIGS. 19-20). ¹¹B NMR spectra were also acquired throughout this process and support the same conclusion. In particular, when all components had been added, a peak with chemical shift characteristic of a tetracoordinate boron emerged (δ −1.41, FIG. 19-20),^{[16]} which is consistent with the PBA anchor forming three boron-oxygen (B—O) bonds, including the oxyanion of the C10P phosphate, as well as the side-chain hydroxyl groups of Ser1 and Ser7. Altogether, the above NMR data support the formation of a stable B1/PBA/C10P ternary complex, wherein calcium supplementation hardly influenced the ³¹P and ¹¹B nuclei surroundings. The MIC, TLC, MS, and NMR experiments presented above are all consistent with B1 functioning via a PBA inducible C10P sequestration mechanism to disrupt the biosynthesis of the cell envelope.

### Spectrum of Activity

We tested B1 against a panel of bacteria in growth medium supplemented with PBA (50 μg/mL) (FIG. 6 and FIG. 25). It inhibited the growth of several Gram-positive pathogenic bacteria, including B. cereus, E. faecalis, Rhodococcus hoagii, as well as a number of Staphylococcus and Streptococcus species. In particular, methicillin-resistant and vancomycin-resistant strains (MRSA and VRE) are similarly susceptible to B1, suggesting that its MOA is orthogonal to wide-spread resistance mechanisms. B1 and its parent compound LspC both showed no activity against Gram-negative bacteria (Escherichia coli and Pseudomonas aeruginosa).

### Boron Dependence Helps Elucidate the Mechanism of CDA Activation

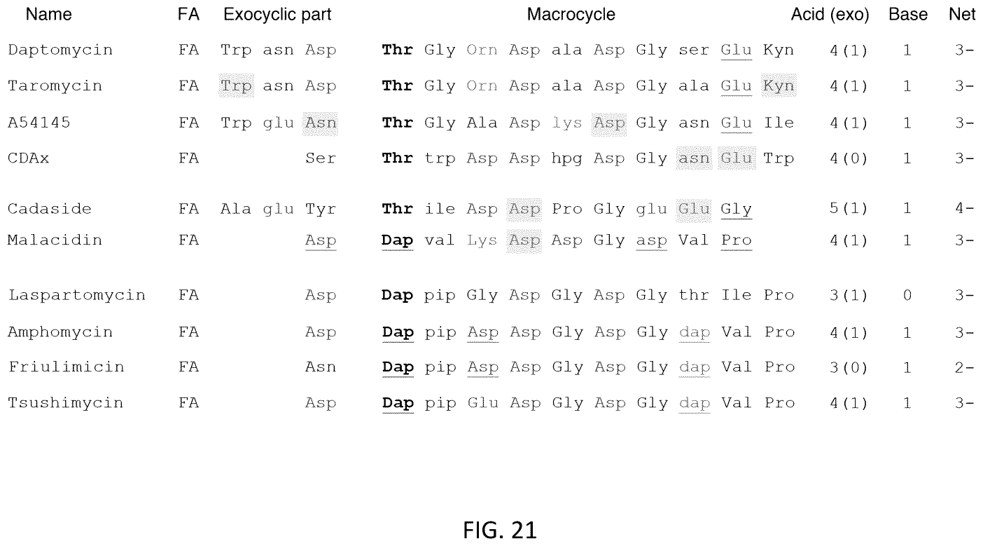

Calcium is thought to induce and secure a CDA in a conformation poised to bind the cell wall biosynthesis intermediate it targets (FIG. 1).^{[10a, 11b]} We report in this manuscript that in LspC, when residues that interact with the anchor (calcium) were replaced by ones that can interact with a boronic acid, dependence on calcium for antimicrobial activity was converted to dependence on boronic acid for the resulting peptides. Specifically, we synthesized and tested a series of four peptides, termed B1 to B4, using either Ser or Hse to replace residue 1 and 7. The subtle differences by a single methylene unit turned out to be a crucial factor in antimicrobial activity. Specifically, whereas B2 was completely inactive, the other three peptides can be activated by PBA. B1 and B3 both behaved exactly as designed, i.e., they were only able to suppress bacterial growth in the presence of PBA, and calcium supplementation no longer affected their activities (entry 4 and 8, FIG. 3). Surprisingly, B4 shows weak antimicrobial activity at 5 mM calcium in the absence of PBA (entry 11, FIG. 3). We speculated that this unexpected activity of B4 may be attributed to one or more of the following. First, the exocyclic residue of CDA (a calcium-dependent antibiotic termed “CDA”) is Ser (FIG. 21).^{[17]} This suggests that even though Asp is a much stronger calcium ligand than Ser/Hse, B4 may interact weakly with calcium to result in partial activation. In addition, certain nonspecific mechanisms may be at play. For example, high calcium concentration and peptides of amphiphilic nature are both known to destabilize the cell envelope.^{[18]} Further studies are required to elucidate the exact antimicrobial mechanism of B4 at this condition.

Mechanistic studies using TLC, MS, and NMR experiments all point to the formation of a stable B1/PBA/C10P ternary complex in the absence of calcium (FIG. 5). These results suggest that PBA alone is sufficient for robust C10P sequestration by B1. We can infer from these observations that the role of the external calcium (Ca2) seen in the LspC crystal structure (FIG. 1B) was indeed replaced by PBA in B1 (FIG. 1C). The fact that each CDA molecule binds two calcium cations inevitably complicates data interpretation in mechanistic studies. Since B1 no longer relied on supplemented calcium for activation, it helped resolve this conundrum and offered an opportunity to decipher the intricate interplay between calcium binding, conformational changes, and substrate sequestration. Simply put, the external calcium (Ca2) is the more important one in LspC; however, additional studies are necessary to confirm whether this applies to all CDAs.

### CONCLUSION

Herein, we report the molecular engineering of a peptide antibiotic, wherein substitution of just two residues in the CDA LspC, those involved in calcium binding, eliminated its dependence on calcium for antibiotic activity. The resulting synthetic LspC analogs instead depend on PBA for antibacterial activity. These peptides are as potent as the parent compound LspC and target the same cell wall biosynthesis intermediate C55P. Results presented herein also shed light on the underlying mechanism of calcium dependence and call for a reexamination of the presumed essential DXDG motif of this family of antibiotics (FIG. 2). Finally, our success in converting the dependence on calcium to boron for an antibiotic point to new possibilities in the design and functional control of drug molecules.

Antibacterial Activity of B1 in Combination with Different Boron Species

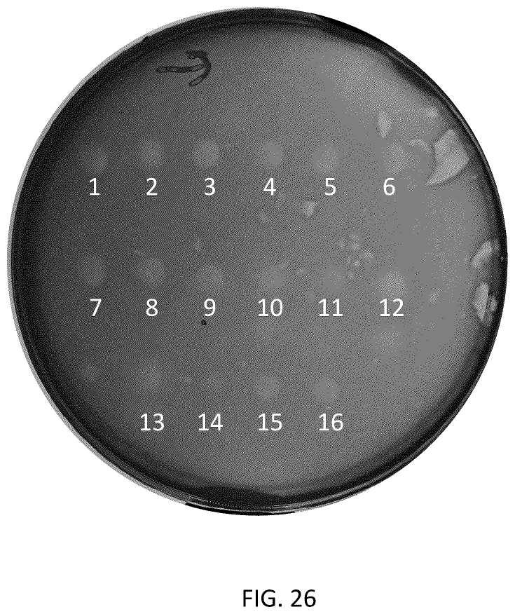

The antimicrobial activities of our peptide (B1) in combination with various boron species were tested by the growth inhibitory zone assay (FIG. 26). B1 was mixed with various boron species at 50 mM each (final concentration). These mixtures were then spotted on a lawn of Bacillus subtilis. After 12 hours of incubation at room temperature, combinations effective at inhibiting bacterial growth would appear as a circular zone. Sixteen boron species (1-16) were tested and all were effective: (1) 4-hydroxylphenylboronic acid, (2) phenylboronic acid, (3) 3-aminophenylboronic acid, (4) 4-aminophenylboronic acid, (5) 4-cyanophenylboronic acid, (6) 4-methoxyphenylboronic acid, (7) 4-pyridylboronic acid, (8) 3-hydroxylphenylboronic acid, (9) 4-fluorophenylboronic acid, (10) 2-formylphenylboronic acid, (11) 4-(methylthio) phenylboronic acid, (12) 4-methoxycarbonyl phenylboronic acid, (13) 3-pyridylboronic acid, (14) 4-tolylboronic acid, (15) 3,5-dimethylphenylboronic acid, (16) 3,5-(bis(trifluoromethyl)phenyl) boronic acid.

It can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

References which are each hereby incorporated in their entirety
[1] V. G. Fowler, A. Jezek, E. S. Spivak, K. Talkington, Urgent, comprehensive federal action needed to stem mortality and medicare costs associated with antimicrobial resistance. Clin. Infect. Dis. 2022, 74, 1107-1111.
[2] a) GBD 2019 Antimicrobial Resistance Collaborators, Global mortality associated with 33 bacterial pathogens in 2019: a systematic analysis for the Global Burden of Disease Study 2019. Lancet 2022, 400, 2221-2248; b) Antimicrobial Resistance Collaborators, Global burden of bacterial antimicrobial resistance in 2019: a systematic analysis. Lancet 2022, 399, 629-655.
[3] AMR Review. Review on Antimicrobial Resistance. https://amr-review.org (accessed 2023 Oct. 4).
[4] T. M. Wood, N. I. Martin, The calcium-dependent lipopeptide antibiotics: structure, mechanism, & medicinal chemistry. MedChemComm 2019, 10, 634-646.
[5] B. M. Hover, et al., Culture-independent discovery of the malacidins as calcium-dependent antibiotics with activity against multidrug-resistant Gram-positive pathogens. Nat. Microbiol. 2018, 3, 415-422.
[6] W. A. Adedeji, The treasure called antibiotics. Ann. Ib. Postgrad. Med. 2016, 14, 56-57.
[7] F. Grein, et al., Ca (2+)-Daptomycin targets cell wall biosynthesis by forming a tripartite complex with undecaprenyl-coupled intermediates and membrane lipids. Nat. Commun. 2020, 11, 1455.
[8] a) D. K. Atchison, W. H. Beierwaltes, The influence of extracellular and intracellular calcium on the secretion of renin. Pflugers Arch. 2013, 465, 59-69; b) Yu, E., Sharma, S. Physiology, Calcium. StatPearls Publishing, Florida, 2023.
[9] a) L. H. J. Kleijn, S. F. Oppedijk, P. Hart, R. M. van Harten, L. A. Martin-Visscher, J. Kemmink, E. Breukink, N. I. Martin, Total synthesis of laspartomycin C and characterization of its antibacterial mechanism of action. J. Med. Chem. 2016, 59, 3569-3574; b) C. Wu, Z. Shang, C. Lemetre, M. A. Ternei, S. F. Brady, Cadasides, calcium-dependent acidic lipopeptides from the soil metagenome that are active against multidrug-resistant bacteria. J. Am. Chem. Soc. 2019, 141, 3910-3919.
[10] a) L. H. J. Kleijn, H. C. Vlieg, T. M. Wood, J. S. Torano, B. J. C. Janssen, N. I. Martin, A high-resolution crystal structure that reveals molecular details of target recognition by the calcium-dependent lipopeptide antibiotic laspartomycin C. Angew. Chem. Int. Ed. 2017, 56, 16546-16549; b) A. Diehl, T. M. Wood, S. Gebhard, N. I. Martin, G. Fritz, The cell envelope stress response of Bacillus subtilis towards laspartomycin C. Antibiotics 2020, 9; c) T. M. Wood, K. Bertheussen, N. I. Martin, The contribution of achiral residues in the laspartomycin family of calcium-dependent lipopeptide antibiotics. Org. Biomol. Chem. 2020, 18, 514-517; d) I. Kotsogianni, T. M. Wood, F. M. Alexander, S. A. Cochrane, N. I. Martin, Binding studies reveal phospholipid specificity and its role in the calcium-dependent mechanism of action of daptomycin. ACS Infect. Dis. 2021, 7, 2612-2619; e) W. V. Curran, R. A. Leese, H. Jarolmen, D. B. Borders, D. Dugourd, Y. C. Chen, D. R. Cameron, Semisynthetic approaches to laspartomycin analogues. J. Nat. Prod. 2007, 70, 447-450.

What is claimed is:

1. An antibiotic of formula (I)
X—Y—Z Formula (I);
wherein:
X comprises a long-chain fatty acid;
Y comprises a linear peptide comprising one, two, or three amino acids;
Z comprises a cyclic peptide comprising nine or ten amino acids; and
wherein the amino acid sequence of the antibiotic is identical or similar to the amino acid sequence of the peptide of a parent calcium dependent antibiotic (CDA) except at least one aspartic acid (Asp) residue or a variant thereof in the amino acid sequence of the peptide of the parent CDA is replaced with a non-Asp residue in the amino acid sequence of Y—Z of the antibiotic of formula (I), wherein the parent CDA comprises daptomycin, taromycin A, taromycin B, A54145, CDA, cadaside A, cadaside B, malacidin A, malacidin B, laspartomycin, amphomycin A, amphomycin B, amphomycin C, amphomycin D, friulimicin A, friulimicin B, friulimicin C, friulimicin D, or a synthetic analogue thereof comprising similar or improved calcium-dependent antibacterial activity.

2. The antibiotic of claim 1, wherein the Asp residue or a variant thereof comprises L-Asp, D-Asp, 3-methoxy-aspartic acid, beta-hydroxy-aspartic acid, or 3-methyl-aspartic acid.

3. The antibiotic of claim 1, wherein the non-Asp residue that replaces the at least one Asp residue or a variant thereof of the parent CDA comprises Ser, Hse, Thr, or Hth.

4. The antibiotic claim 1, wherein X comprises a long-chain fatty acid identical to the fatty acid chain of the parent CDA or a synthetic analogue thereof.

5. The antibiotic of claim 1, wherein X comprises a long-chain fatty acid comprising a carbon number about 80% to about 120% as the carbon number of the fatty acid chain of the parent CDA.

6. The antibiotic claim 1, wherein X comprises —(CO)(CH₂)₁₄ (CH₃), —(CO)(CH)₂(CH₂)₉(CH)(CH₃)₂, —(CO)(CH₂)₈ (CH₃), —(CO)(CH)₄(CH₂)₂(CH₃), —(CO)(CH)₄(CH)(CH₃)(CH₂)(CH₃), —(CO)(CH₂)₆(CH)(CH₃)₂, —(CO)(CH₂)₆(CH)(CH₃)(CH₂)(CH₃), —(CO)(CHOCH)(CH₂)₂(CH₃), —(CO)(CH)₄(CH₂)₂(CH)(CH₃)₂, —(CO)(CH)₄(CH₂)₂(CH)(CH₃)(CH₂)(CH₃), —(CO)(CH₂)(CH)₂(CH₂)₆(CH)(CH₃)₂, —(CO)(CH₂)(CH)₂(CH₂)₇(CH)(CH₃)₂, —(CO)(CH₂)(CH)₂(CH₂)₅(CH)(CH₃)(CH₂)(CH₃), or —(CO)(CH₂)(CH)₂(CH₂)₇(CH)(CH₃)(CH₂)(CH₃).

7. The antibiotic of claim 1, wherein amino acid sequence of Y comprises one, two, or three non-Asp residues.

8. The antibiotic of claim 7, wherein amino acid sequence of Y is identical or similar to Trp-Asn-Xaa, Trp-Glu-Asn, Ala-Glu-Tyr, or Xaa, wherein each Trp comprises Trp or 6-chloro-tryptophan, wherein each Asn comprises Asn, D-Asn, or 3-hydroxyl-L-asparagine, wherein each Glu comprises D-Glu, wherein each Xaa comprises a non-Asp residue.

9. The antibiotic of claim 8, wherein amino acid sequence of Y is identical to Ser, Hse, Thr, or Hth.

10. The antibiotic of claim 1, wherein amino acid sequence of Z is identical to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

11. The antibiotic of claim 1, wherein amino acid sequence of Z is identical to SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18.

12. The antibiotic of claim 1, wherein the amino acid sequence of Z is at least about 80%, about 85%, about 90%, about 95%, or about 100% identical or similar to SEQ ID NO: 15 or SEQ ID NO: 16.

13. The antibiotic of claim 1, wherein amino acid sequence of Y—Z is identical or similar to SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29.

14. The antibiotic of claim 1, wherein amino acid sequence of Y—Z is at least about 80%, about 85%, about 90%, about 95%, or about 100% identical or similar to SEQ ID NO: 26, SEQ ID NO: 27, SEQ ID NO: 28, or SEQ ID NO: 29.

15. The antibiotic of claim 1, wherein the antibiotic comprises a formula of

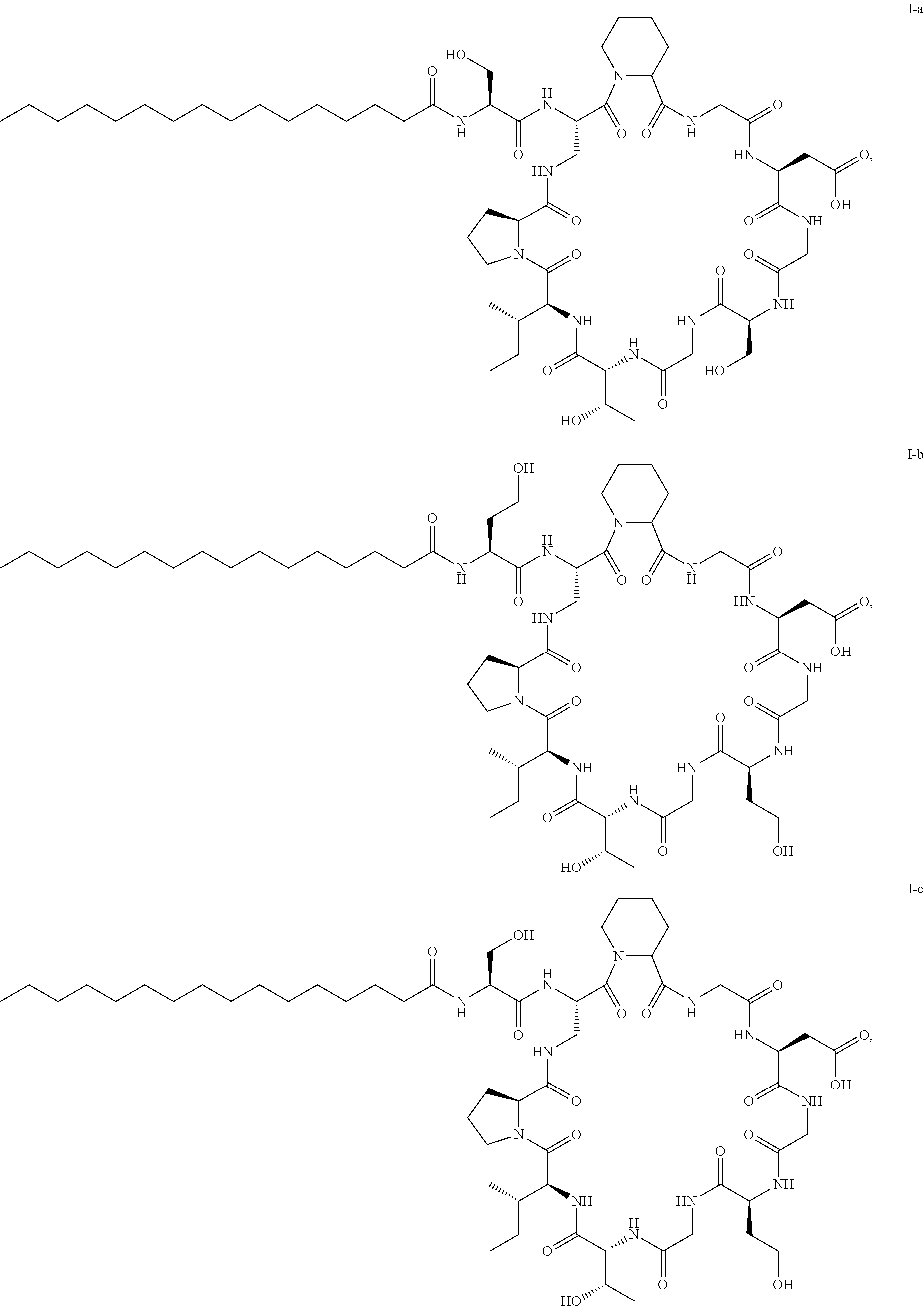

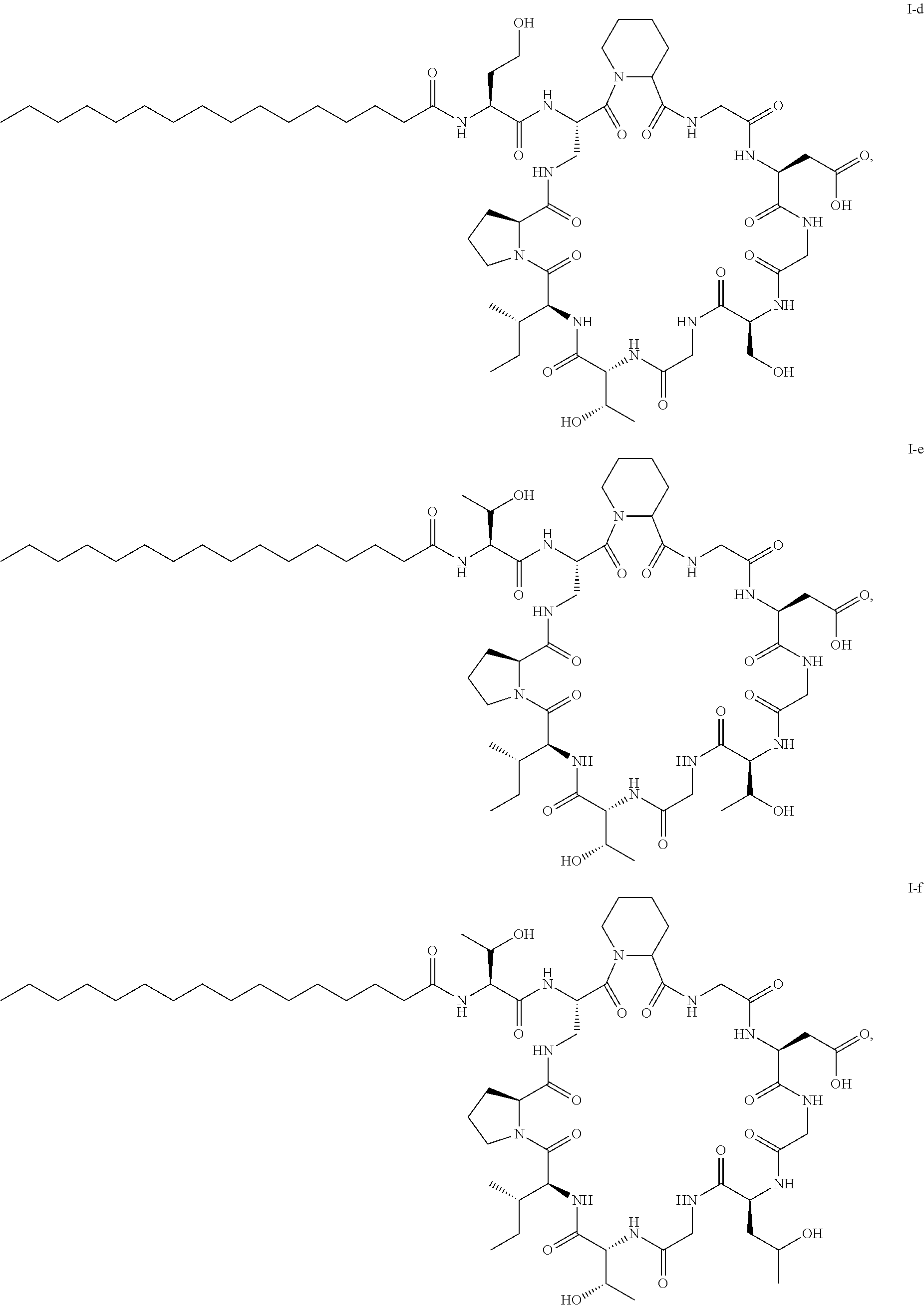

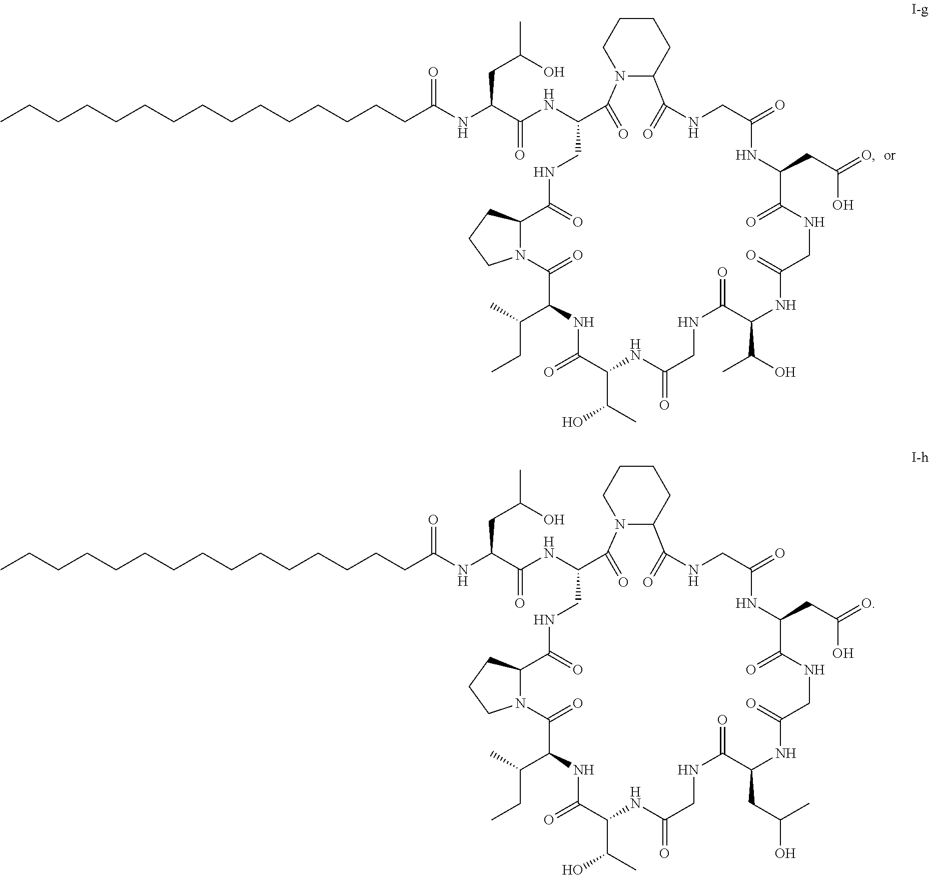 [SMILES: CCCCCCCCCCCCCCCC(=O)N[C@@H](CC(C)O)C(=O)N[C@H]1CNC(=O)[C@@H]2CCCN2C(=O)[C@H]([C@@H](C)CC)NC(=O)[C@@H]([C@H](C)O)NC(=O)CNC(=O)[C@H](C(C)O)NC(=O)CNC(=O)[C@H](CC(=O)O)NC(=O)CNC(=O)C2CCCCN2C1=O ; CCCCCCCCCCCCCCCC(=O)N[C@@H](CC(C)O)C(=O)N[C@H]1CNC(=O)[C@@H]2CCCN2C(=O)[C@H]([C@@H](C)CC)NC(=O)[C@@H]([C@H](C)O)NC(=O)CNC(=O)[C@H](CC(C)O)NC(=O)CNC(=O)[C@H](CC(=O)O)NC(=O)CNC(=O)C2CCCCN2C1=O]

16. A method of preparation of the antibiotic of claim 1 comprising the step of performing solid-phase peptide synthesis (SPSS) using amino acid building blocks compatible with the fluorenylmethoxycarbonyl (Fmoc) chemistry or the tert-butoxycarbonyl (BOC) chemistry.

17. The method of claim 16 wherein the step of performing SPSS comprises the steps of
a) loading a first amino acid building block on a resin;
b) attaching a first peptide to the first amino acid building block loaded on the resin of step a;
c) attaching a long-chain fatty acid to the first peptide linked to the first amino acid building block loaded to the resin of step b;
d) attaching a second peptide to the first peptide at amino acid residue of the first peptide to form a linear form of the antibiotic of claim 1;
e) removing the linear form of the antibiotic of claim 1 of step d from the resin;
f) circularizing the linear form of the antibiotic of claim 1 of step e to form the antibiotic of claim 1; and
g) removing the protecting groups on the amino acid building blocks of the crude the antibiotic of claim 1 of step f to obtain the antibiotic of claim 1 of the present invention.

18. A method of inhibiting bacterial growth in a bacterial culture using the antibiotic of claim 1 comprising the step of administration of the antibiotic to the bacterial culture comprising at least a bacteria colony.

19. The method of claim 18, wherein the bacteria comprise Gram-positive bacteria, drug-resistant bacteria, or a combination thereof.

20. The method of claim 18 further comprising the step of administration of a boron atom.

21. The method of claim 20, wherein the boron atom comprises phenylboronic acid (PBA), 4-hydroxylphenylboronic acid, 3-aminophenylboronic acid, 4-aminophenylboronic acid, 4-cyanophenylboronic acid, 4-methoxyphenylboronic acid, 4-pyridylboronic acid, 3-hydroxylphenylboronic acid, 2-formylphenylboronic acid, 4-(methylthio) phenylboronic acid, 4-methoxycarbonyl phenylboronic acid, 3-pyridylboronic acid, 4-tolylboronic acid, 3,5-dimethylphenylboronic acid, 3,5-(bis(trifluoromethyl)phenylf) boronic acid, 4-fluorophenylboronic acid, or a combination thereof.

22. A method of treatment of bacterial infection in a subject comprising the step of administration of a therapeutically effective amount of the antibiotic of claim 1 to the subject.

23. The method of claim 22, wherein the bacterial infection comprises infection by Gram-positive bacteria, drug-resistant bacteria, or a combination thereof.

24. The method of claim 22, further comprises the step of administration of a therapeutically effective amount of a boron atom composition comprising boron atom.

25. The method of claim 24, wherein the boron atom composition comprises phenylboronic acid (PBA), 4-hydroxylphenylboronic acid, 3-aminophenylboronic acid, 4-aminophenylboronic acid, 4-cyanophenylboronic acid, 4-methoxyphenylboronic acid, 4-pyridylboronic acid, 3-hydroxylphenylboronic acid, 2-formylphenylboronic acid, 4-(methylthio) phenylboronic acid, 4-methoxycarbonyl phenylboronic acid, 3-pyridylboronic acid, 4-tolylboronic acid, 3,5-dimethylphenylboronic acid, 3,5-(bis(trifluoromethyl)phenylf) boronic acid, 4-fluorophenylboronic acid, or a combination thereof.
